(19) United States
(12) Patent Application Publication
(10) Pub. No.: US 2017/0333572 A1
(43) Pub. Date: Nov. 23, 2017

(54) RNA GUIDED ERADICATION OF HUMAN JC VIRUS AND OTHER POLYOMAVIRUSES

(71) Applicant: Temple University of Commonwealth System of Higher Education, Philadelphia, PA (US)

(72) Inventors: Kamel Khalili, Bala Cynwyd, PA (US); Wenhui Hu, Cherry Hill, NJ (US); Hassen Wollebo, Philadelphia, PA (US)

(21) Appl. No.: 15/523,272

(22) PCT Filed: Oct. 30, 2015
(86) PCT No.: PCT/US15/58351

§ 371 Date: Apr. 28, 2017

Related U.S. Application Data
(60) Provisional application No. 62/072,927, filed on Oct. 30, 2014.
(60) Provisional application No. 62/169,390, filed on Jun. 1, 2015.
(60) Provisional application No. 62/169,638, filed on Jun. 2, 2015.

(51) Int. Cl. A61K 48/00; C12N 9/22; C12N 15/113; C12N 2310/20

(52) U.S. Cl. CPC A61K 48/005; C12N 15/1131; C12N 9/22; C12N 2310/20

(57) ABSTRACT

The present invention includes methods and compositions for elimination of polyomaviruses, such as John Cunningham Virus (JVC), from host cells, and the treatment of polyomavirus related diseases, such as progressive multifocal leukoencephalopathy (PML). The compositions include isolated nucleic acid sequences comprising a CRISPR-associated endonuclease and a guide RNA, wherein the guide RNA is complementary to a target sequence in a polyomavirus.

### STATEMENT REGARDING FEDERALLY SPONSORED RESEARCH

This invention was made with U.S. government support under grant numbers NIH R01 NS087971 to Kamel Khalili and Wenhui Hu awarded by the National Institutes of Health. The U.S. government may have certain rights in the invention.

The Sequence Listing associated with this application is filed in electronic format via EFS-Web and hereby incorporated by reference into the specification in its entirety. The name of the text file containing the Sequence Listing is 0392-7 Temple JCV PCT SeqLst 10 30 2015_ST25. The size of the text file is 8 KB, and the text file was created on Oct. 30, 2015.

### FIELD OF THE INVENTION

The present invention relates to compositions that specifically cleave target sequences in polyomaviruses, for example, human neurotropic polyomavirus such as John Cunningham virus. Such compositions, which can include a Clustered Regularly Interspaced Short Palindromic Repeats (CRISPR) associated endonuclease, and one or more specific guide RNA sequences, can be administered to a subject having a neurotropic polyomavirus infection.

### BACKGROUND

The human neurotropic polyomavirus, John Cunningham virus (JCV) is the etiological agent of a fatal demyelinating disease, progressive multifocal leukoencephalopathy (PML). Lytic infection of JCV in glial cells of the central nervous system (CNS) results in the death of oligodendrocytes, the cells that are responsible for the production of myelin sheaths in the brain. This leads to a broad range of mild to severe neurological disturbances and eventually death (Berger, 2011). There are a number of predisposing factors to PML but all involve some level of impairment of the immune system.

While the disease was first recognized as a rare disorder predominantly seen in patients with lymphoproliferative and myeloproliferative disorders (Astrom, et al., 1958), the onset of the AIDS pandemic greatly increased the incidence of PML. HIV-1 infection and AIDS remain the most frequent immunodeficiency setting for the reactivation of JCV, accounting for approximately 80% of PML cases (Tavazzi, et al. 2012). Immunosuppresive therapies have become another trigger of active JCV infection. Treatments for autoimmune disorders such as multiple sclerosis and rheumatoid arthritis, with new therapeutic immunomodulatory monoclonal antibodies, including natalizumab (Chakley and Berger, 2013) efalizumab (Schwab, et al., 2102) and rituximab Clifford, et al.,2011) ,is recognized as a predisposing factor for PML (Nagayama, et al., 2013).

JCV is a member of the polyomavirus family of viruses, whose genome is comprised of double-stranded circular DNA of 5.1 kb in size, which produce two classes of proteins at the early phase of viral infection, i.e. before DNA replication, and the late phase of the infection cycle (DeCaprio, et al., 2013). A bi-directional coding sequence positioned between the early and late genes is responsible for viral gene expression and contains the origin of viral DNA replication. The viral early protein, large T-antigen (T-Ag) and a family of smaller sized T-Ag proteins are produced by alternative splicing, and have a regulatory role in orchestrating the virus during its replication cycle. The large T-antigen, in particular, is responsible for initiation of viral DNA replication and stimulation of viral late gene transcription, and thus is critical for all aspects of the viral life cycle. In addition, JCV T-Ag exhibits transforming ability in cell culture and its expression in animal models, in the absence of the other viral proteins, induces tumors of neural origin (for review see White and Khalili, 2004). Tag binds to several cellular proteins such as p53 and pRb, and dysregulates proliferation of cells, thus potentially leading to transformation of cells and formation of tumors in several animal models. The late proteins are the viral capsid proteins VP1, VP2, and VP3 and a small regulatory protein known as agnoprotein Khalili, et al., 2005). Seroepidemiological studies have shown that JCV infection is very common in populations throughout the world and initial infection usually occurs during childhood (White and Khalili, 2011). The high seroprevalence of JCV infection and the rarity of PML suggest that the immune system is able to maintain the virus in a persistent asymptomatic state, since altered immune function appears to underlie all conditions that predispose to PML.

A number of treatment options have been applied to PML, largely without success (Tavazzi, et al. 2012). Different approaches have targeted various points in the viral life cycle such as entry and replication. Since interaction between JCV and the serotonin 2A receptor (5-HT2AR) has been reported to be required for viral entry (Elphick, et al., 2004) risperidone, which binds 5HT2AR, has been studied but found to have no effect (Chapagain, et al., 2008). Small molecule inhibitors of viral replication such as cidofovir have been tested in vitro and in vivo but have yielded conflicting results (Andrei, et al., 1997, Hou and Major, 1998). Clearly alternative strategy options are urgently required for treatment of this fatal demyelinating disease.

New strategies that target the JCV viral genome for eradication are particularly attractive. This strategy can effectively target both actively replicating virus and persistent virus, in which the virus either remains in a dormant state or in which viral proteins are either not expressed or are produced at very low levels. Recent advances in engineered nuclease technology have raised the prospect that this therapeutic strategy may soon be possible in the clinic. Examples are zinc-finger nucleases (ZFN), transcription activator-like effector nucleases (TALEN) and more recently clustered regulatory interspaced short palindromic repeat (CRISPR)-associated 9 (Cas9) (Gaj, et al., 2013).

In particular, tools and techniques based on CRISPR/Cas9 DNA editing systems offer unprecedented control over genome editing (Mali, et al., 2013, Hsu, et al., 2014). The CRISPR/Cas9 system was developed from the adaptive immune system of bacteria and archaea, and uses a short guide RNA (gRNA) to direct the cleavage of specific nucleic acids by a Cas9 endonuclease (Bhaya, et al., 2011). The cleavage, usually a blunt ended double-strand cut, usually causes deletions, insertions, and excisions of stretches of DNA, caused by defective DNA repair.

CRISPR/Cas9 DNA editing systems have been used to inactivate the oncogenic human papilloma genes E6 and E7 in cervical carcinoma cells (Kennedy, et al., 2014) and to facilitate clearance of intrahepatic hepatitis B genome templates in vivo (Lin, et al., 2014). More recently, it was reported that CRISPR/Cas9 can be used to eliminate HIV-1 provirus from latently infected cells and prevent new HIV-1 infection (Hu, et al., 2014). Unfortunately, no systems have yet been developed to target JCV with a gRNA-guided endonuclease attack. There is a great need for CRISPR/endonuclease compositions and methods for cleaving specific targets in the JCV genes, destroying the integrity of the JCV genome, and thus eliminating JCV from host cells.

### SUMMARY

The present invention provides a composition for use in eliminating JCV from a host cell infected with JCV. The composition includes at least one isolated nucleic acid sequence encoding a Clustered Regularly Interspaced Short Palindromic Repeat (CRISPR)-associated endonuclease, and at least one guide RNA (gRNA) having a spacer sequence complementary to a target sequence in a JCV DNA. The preferred target sequences for the gRNAs are in the large T antigen (T-Ag) gene of the JCV DNA, especially the TM1, TM2, and TM3 regions of T-Ag.

The present invention also provides a method of eliminating JCV from a host cell infected with JCV. The method includes the steps of treating the host cell with a composition comprising a CRISPR-associated endonuclease, and at least one gRNA having a spacer sequence complementary to a target sequence in a JCV DNA; and eliminating the JCV from the host cell.

The present invention further provides a vector composition for use in eliminating JCV from a host cell infected with JCV. The vector composition includes at least one isolated nucleic acid sequence encoding a CRISPR-associated endonuclease, and at least one gRNA having a spacer sequence complementary to a target sequence in a JCV DNA. The isolated nucleic acid sequences are included in at least one expression vector for inducing expression of the CRISPR-associated endonuclease and at least one gRNA in a host cell.

The present invention still further provides a method of preventing JCV infection of cells of a patient at risk of JCV infection. The method includes the steps of determining that a patient is at risk of JCV infection; exposing patient cells to an effective amount of an expression vector composition including an isolated nucleic acid encoding a CRISPR-associated endonuclease, and at least one isolated nucleic acid, encoding at least one gRNA, which includes a spacer sequence complementary to a target sequence in a JCV DNA; stably expressing the CRISPR-associated endonuclease and gRNAs in the cells of the patient; and preventing JCV infection of the patient's cells.

The present invention also provides a pharmaceutical composition for the elimination of JCV from the cells of a mammalian subject. The pharmaceutical composition includes at least one isolated nucleic acid sequence encoding a CRISPR-associated endonuclease, and at least one isolated nucleic acid sequence encoding at least one gRNA having a spacer sequence complementary to a target sequence in a JCV DNA. In a preferred embodiment, the isolated nucleic acid sequences are included in at least one expression vector.

The present invention further provides a method of treating a subject having a LCV related disorder, such as progressive multifocal leukoencephalopathy, including the step of administering, to the subject, an effective amount of the previously stated pharmaceutical composition.

The present invention still further provides a kit for the treatment or prophylaxis of JCV infection, including a measured amount of one or more of the previously stated compositions including CRISPR-associated endonucleases and gRNAs complementary to target sequences in a JCV DNA. The kit also includes one or more items such as instructions for use, sterile containers, and syringes.

The present invention also provides a method of eliminating polyomaviruses other than JCV from host cells of the viruses. The method includes the steps of treating the host cells with a composition including a CRISPR-associated endonuclease, and at least one gRNA having a spacer sequence complementary to a target sequence in a polyomavirus DNA; and eliminating the polyomavirus from the host cells. Preferably, the target sequence is situated in the region encoding the large T antigen of the specific polyomavirus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other advantages of the present invention are readily appreciated as the same becomes better understood by reference to the following detailed description when considered in connection with the accompanying drawings wherein:

FIGS. 1A and 1B show the design of guide RNAs for CRISPR/Cas9 targeting of JCV. FIG. 1A shows three gRNAs (TM1, TM2 and TM3) that were designed at different positions within the coding region for JCV T-Ag (T) as shown. The T-Ag coding region begins at nucleotide (nt) 5013 of the 5130 nt circular Mad-1 JCV genome (NCBI Reference Sequence: NC_001699.1; Frisque et al, 1984) and proceeds anticlockwise to nt 2603. FIG. 1B shows the sequence of the JCV genome at each of the three targeted sites (bold and red highlight) is given. Note that the sequence of the top strand is clockwise and antisense to the coding region of T-Ag since T-Ag is transcribed anticlockwise and shown on the bottom (sense strand). The position of the protospacer adjacent motif (PAM) sequence is shown in blue italics;

FIGS. 2A-2C show that expression of gRNAs m1 and m2 caused reduction of T-Ag expression and T-Ag-stimulated JCV late gene expression in TC620 cells transfected with T-Ag and Cas9. FIG. 2A: TC620 cells were transfected with JCV_{L}-LUC reporter plasmid and expression plasmid for Cas9 with and without expression plasmids for T-Ag and each of the gRNAs shown in FIG. 1, alone or in combination as indicated. Cells were harvested and luciferase activity was assayed as described in Example 1. Activities were normalized to cells transfected with reporter plasmid alone (lane 1). FIG. 2B: Cell extracts referred to in FIG. 2A were analyzed by Western blot for expression of T-Ag, Cas9 and α-tubulin (loading control). FIG. 2C: The Western blots shown in FIG. 2B were quantified using Bio-Rad Quantity One software and shown as a histogram normalized to T-Ag alone (lane 2);

FIGS. 3A-3D show that a clonal derivative of SVGA expressing Cas9 and gRNA ml have reduced capacity to support JCV infection. FIG. 3A: SVGA cells were transfected with Cas9 or Cas9 plus gRNA m1, and stable clonal cell lines were selected. Three clones were selected and assayed for JCV infection (moi=1, 7 days): one clone with Cas9 alone and two with Cas9 plus gRNA m1 (clones 8 and 10) relative to parental SVGA cells. Viral infection was assessed by Western blot for VP1 and agnoprotein with α-tubulin as a loading control. FIG. 3B: Virus in the culture supernatants from the experiment in FIG. 3A were quantified using QPCR. FIG. 3C: SVGACas9 and SVGACas9m1c8 were assayed for Cas9 expression by Western blot with [3-tubulin used as a loading control. FIG. 3D: TC620 cells were transfected with expression plasmid for FLAG-tagged Cas9 and immunocytochemistry performed with anti-FLAG antibody as described in Materials and Methods. Nuclei were labeled with 4′,6-diamidino-2-phenylindole (DAPI);

FIGS. 4A-4C show a direct demonstration of T-Ag gene cleavage after transient transfection of Cas9 and JCV-specific gRNA. Mouse BsB8 and hamster HJC-2 cells, which both contain an integrated TAg gene, were transfected with expression plasmid for Cas9 and the gRNAs in various combinations as indicated. Genomic DNA was amplified using JCV-specific primers. FIG. 4A shows a diagram of the T-Ag gene, indicating the positions of the PCR primers, the expected cleavage points, and the expected lengths of resulting regions of the T-Ag gene. FIG. 4B: The T-Ag gene from transfected BsB8 cells was amplified by PCR, electrophoresed on an agrose gel, and labeled with ethidium bromide. The image is inverted for clarity of presentation. FIG. 4C: The T-Ag gene from transfected HJC-2 cells was amplified by PCR, electrophoresed on an agarose gel and labeled with ethidium bromide. The image is inverted for clarity of presentation;

FIG. 5 depicts primers for targeting three different motifs of T-antigen;

FIGS. 6A and 6B show that stable derivatives of HJC-2 cells expressing doxycycline-inducible Cas9 exhibit inDel mutations of the T-Ag gene upon transduction with lentiviruses expressing JCV-specific gRNAs and doxycycline induction. FIG. 6A: HJC-2 cells expressing doxycycline-inducible Cas9 were transduced with lentiviruses expressing JCV-specific gRNAs and treated with and without doxycycline as described in Example 1. Total genomic DNA was extracted and regions of the T-Ag were amplified by PCR, cloned into pCR4-TOPOTA vector and sequenced. FIG. 6B: The Surveyor assay was used to detect the presence of mutations in PCR products derived from HJC-2 cells expressing Cas9 and transduced by lentiviral vectors for each of the gRNAs (m1, m2 and m3). PCR products were denatured and hybridized by gradual cooling as described in Example 1. Hybridized DNA was digested with SURVEYOR nuclease to cut heteroduplex DNA and samples were resolved on a 2% agarose gel together with equal amounts of control samples; the control samples were treated in parallel but derived from HJC-2 cells expressing Cas9 but not transduced by lentiviral vectors encoding gRNAs (ml Con, m2 Con, m3 Con);

FIGS. 7A and 7B show that stable derivatives of HJC-2 cells expressing doxycycline-inducible Cas9 exhibit ablation of T-Ag expression upon transduction with lentiviruses expressing JCV-specific gRNAs and doxycycline induction. FIG. 7A: Western blot showing the expression of Cas9 and T-Ag. JC-2 stable cell clones expressing doxycycline-inducible Cas9 were transduced with lentiviral vectors for each of the three gRNAs as described in Example 1. After 24 hours, the transduced cells were treated with and without 2 μg/ml doxycycline and after another 48 hours harvested and expression of T-Ag and Cas9 analyzed by Western blot with a-tubulin as a loading control. FIG. 7B shows a quantification of the Western blot;

FIGS. 8A-8E show that stable derivatives of HJC-2 cells expressing doxycycline-inducible Cas9 exhibit reduced colony formation upon transduction with lentiviruses expressing JCV-specific gRNAs and doxycycline induction. HJC-2 cells expressing doxycycline-inducible Cas9 were transduced with lentiviruses expressing m1, m2 and m3 gRNAs in various combinations as indicated, plated, treated with or without doxycycline and assessed for colony formation. Results are shown as histograms of the total numbers of colonies obtained with the error bars representing one standard deviation calculated from replicate colony counts. FIG. 8A shows results for the combination m1+m2, FIG. 8B shows results for the combination m1+m3, FIG. 8C shows results for the combination m2+m3, and FIG. 8D shows results for the combination m1+m2+m3. FIG. 8E shows a photograph of a representative experiment showing two methylene blue stained dishes from the experiment summarized in FIG. 8A.

FIGS. 9A-9C show that stable derivatives of SVG-A cells expressing Cas9 and gRNAs show no InDel mutations in off target genes. The SURVEYOR assay was used to detect the presence of mutations in PCR products derived from SVG-A cells expressing Cas9 and gRNAs ml (complementary to SEQ ID NO: 1, FIG. 9A), m2 (complementary to SEQ ID NO: 5, FIG. 9B) and m3 (complementary to SEQ ID NO: 9, FIG. 9C). Human cellular genes with the highest degree of homology to each motif were identified by BLAST search at the NCBI website (http://www.ncbi.nlm.nih.gov/). For each motif, PCR product was amplified from the top three genes with the highest degree of homology and examined for InDel mutations using the SURVEYOR assay as described in Example 1. Amplification of T-Ag was the positive control in each figure. FIG. 9A: For motif m1, amplification was of M12 (NM_017821, human RHBDL2 rhomboid, veinlet-like 2 (Drosophila), Gene ID: 54933, NCBI Ref Seq: NC_000001.11, >gi|568815597:c38941830-38885806), M17 (NM_001243540, human KIAA1731NL, Gene ID:100653515, NCBI Ref Seq: NC_000017.11, >gi|568815581:78887721-78903217), and M19 (NM_016252, human BIRC6 baculoviral IAP repeat containing 6, Gene ID: 57448, NCBI Ref Seq: NC_000002.12); FIG. 9B: For motif m2, amplification was of M21 (NM_012090, human MACF1 microtubule-actin crosslinking factor 1, Gene ID: 23499, NCBI Ref Seq: NC_000001.11, >gi|568815597:39084167-39487138), M23 (NM_005898, human CAPRIN1 cell cycle associated protein 1, Gene ID: 4076, NCBI Ref Seq: NC_000011.10, >gi|568815587:34051683-34102610), M24 (NM_024562, human TANGO6 transport and Golgi organization 6 homolog (Drosophila), Gene ID: 79613, NCBI Ref Seq: NC_000016.10, >gi|568815582:68843553-69085482); FIG. 9C: For motif m3, amplification was of M31 (NM_001048194, human RCC1 regulator of chromosome condensation 1, Gene ID: 1104, NCBI Ref Seq: NC_000001.11, >gi|568815597:28505943-28539196), M32 (NM_004673, human ANGPTL1 angiopoietin-like 1, Gene ID: 9068, NCBI Ref Seq: NC_000001.11, >gi|568815597:c178871353-178849535), M33 (NM_174944, human TSSK4 testis-specific serine kinase 4, Gene ID: 283629, NCBI Ref Seq: NC_000014.9, >gi|568815584:24205530-24208248).

### DETAILED DESCRIPTION

The present invention represents the first application of CRISPR technology to the problem of active, latent, and potential infection by JCV. CRISPR technology, unlike the alternative ZFN and TALEN technologies of the prior art, is easily tailored for specific targets, and is multiplexible. The CRISPR compositions and methods of the present invention are effective at eliminating JCV infection from host cells and protecting host cells from future infection.

Compositions and Methods for Polvomavirus Elimination and Prevention of Infection.

The RNA-guided CRISPR biotechnology adapts genome defense mechanisms of bacteria, wherein CRISPR/Cas loci encode RNAguided adaptive immune systems against mobile genetic elements (viruses, transposable elements and conjugative plasmids).

CRISPR clusters encode spacers, the sequences complementary to target sequences (“protospacers”) in a viral nucleic acid, or another nucleic acid to be targeted. CRISPR clusters are transcribed and processed into mature CRISPR (Clustered Regularly Interspaced Short Palindromic Repeats) RNA (crRNA). CRISPR clusters also encode CRISPR associated (Cas) proteins, which include DNA endonucleases. The crRNA binds to target DNA sequence, whereupon the Cas endonuclease cleaves the target DNA at or adjacent to the target sequence.

One useful CRISPR system includes the CRISPR associated endonuclease Cas9. Cas9 is guided by a mature crRNA that contains about 20-30 base pairs (bp) of spacer and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the target sequence on the target DNA. Cas9 recognizes a trinucleotide (NGG) photospacer adjacent motif (PAM) to decide the cut site (the 3rd nucleotide from PAM). The crRNA and tracrRNA can be expressed separately or engineered into an artificial chimeric small guide RNA (sgRNA) via a synthetic stem loop (AGAAAU) to mimic the natural crRNA/tracrRNA duplex. Such sgRNAs, can be synthesized or in vitro transcribed for direct RNA transfection, or they can be expressed in situ, e.g. from U6 or H1-promoted RNA expression vectors. The term “guide RNA” (gRNA) will be used to denote either a crRNA:tracrRNA duplex or an sgRNA. It will be understood the term “gRNA complementary to” a target sequence indicates a gRNA whose spacer sequence is complementary to the target sequence.

The compositions of the invention include nucleic acids encoding a CRISPR-associated endonuclease for example, Cas9, and at least one gRNA complementary to a target sequence in a polyomavirus, e.g., JCV.

In preferred embodiments of the present invention, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophiles; Psuedomonas aeruginosa, Escherichia coli, or other sequenced bacteria genomes and archaea, or other prokaryotic microogranisms. Alternatively, the wild type Streptococcus pyrogenes Cas9 sequence can be modified. Preferably, the nucleic acid sequence is be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins).

The Cas9 nuclease sequence can be a mutated sequence. For example the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR22 can potentially decrease the frequency of unwanted InDel mutations from off-target double-stranded breaks.

In some embodiments, compositions of the invention can include a CRISPR-associated endonuclease polypeptide encoded by any of the nucleic acid sequences described above. Polypeptides can be generated by a variety of methods including, for example, recombinant techniques or chemical synthesis. Once generated, polypeptides can be isolated and purified to any desired extent by means well known in the art. For example, one can use lyophilization following, for example, reversed phase (preferably) or normal phase HPLC, or size exclusion or partition chromatography on polysaccharide gel media such as Sephadex G-25. The composition of the final polypeptide may be confirmed by amino acid analysis after degradation of the peptide by standard means, by amino acid sequencing, or by FAB-MS techniques.

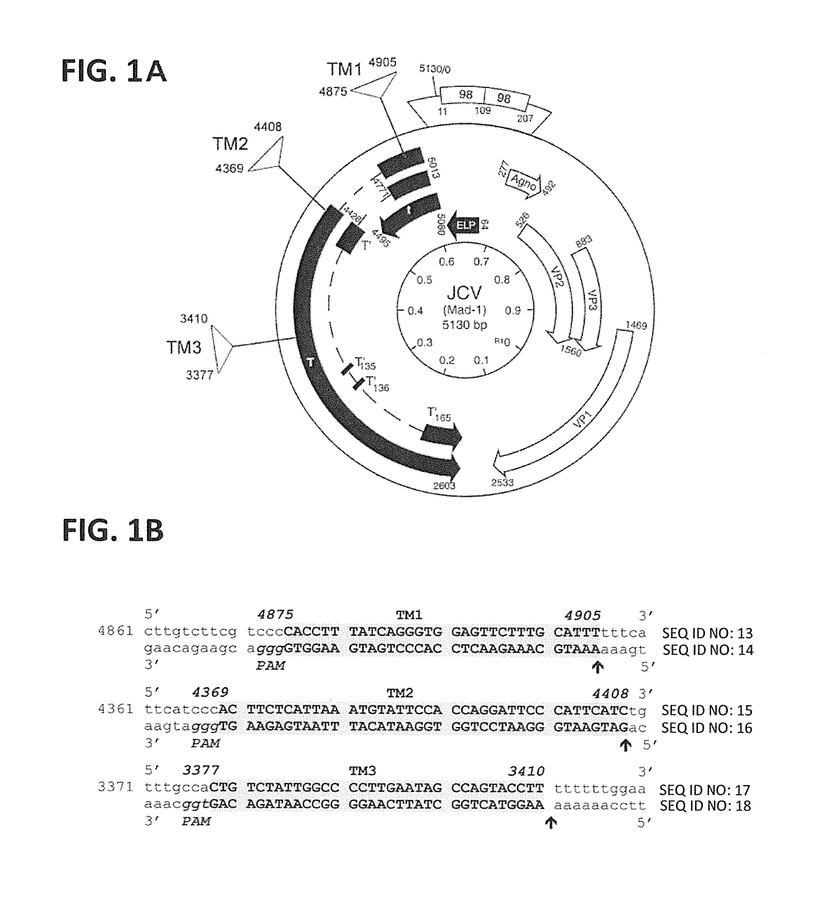

In exemplary embodiments, the present invention includes an engineered CRISPR system including Cas9 and one or more gRNAs complementary to a JCV T-antigen sequence. An exemplary JCV genome sequence is the Mad-1 strain, NCBI reference sequence, GenBank number: NC_001699.1, public GI (Frisque et al, 1984). In the Mad 1 strain, the T-Ag coding region begins at nucleotide (nt) 5013 of the 5130 nt circular Mad-1 JCV genome. Exemplary gRNA spacer sequences are complementary to the TM1, TM2 or TM3 regions JCV T-antigen sequence. The structural organization of Mad-1 JCV is shown in FIG. 1A. Nucleotide sequences corresponding to TM1, TM2, and TM3 are shown in FIG. 1B, and target sequences for gRNAs are shown in bold capital type. Target sequences can extend from approximately 20 to 40 or more nts in length. It will be understood that, in different strains of JCV, or in mutational variants, sequences homologous to TM1, TM2, and TM3 can be readily identified by well known sequencing and genomics techniques.

An exemplary target sequence in TM1 includes SEQ ID NO: 1, or its complement on the antiparallel strand, SEQ ID NO: 2. The PAM sequence in each strand (shown in lower case bold) can be included in the target sequence, so that the target sequences can include SEQ ID NO: 3 or its complement on the antiparallel strand, SEQ ID NO: 4. A gRNA complementary to TM1, designated gRNA m1, can therefore include a spacer sequence complementary to SEQ ID NO: 1, SEQ ID NO: 2, SEQ ID NO: 3; or SEQ ID NO: 4.

The nucleotide sequences are as follows:

(SEQ ID NO: 1)
AAATGCAAAGAACTCCACCCTGATGAAGGTG
(SEQ ID NO: 2)
AAATGCAAAGAACTCCACCCTGATGAAGGTGGGG
(SEQ ID NO: 3)
CACCTTTATCAGGGTGGAGTTCTTTGCATTT
(SEQ ID NO: 4)
CCCCACCTTTATCAGGGTGGAGTTCTTTGCATTT

An exemplary target sequence in TM2 includes SEQ ID NO: 5, or its complement on the antiparallel strand, SEQ ID NO: 6. The PAM sequence in each strand can also be included in the target sequence, so that the target sequences can include SEQ ID NO: 7 or its complement on the antiparallel strand, SEQ ID NO: 8. A gRNA complementary to TM2, designated gRNA m2, can therefore include a spacer sequence complementary to SEQ ID NO:5, SEQ ID NO: 6, SEQ ID NO: 7; or SEQ ID NO: 8.

The nucleotide sequences are as follows:

(SEQ ID NO: 5)
GATGAATGGGAATCCTGGTGGAATACATTTAATGAGAAGT
(SEQ ID NO: 6)
GATGAATGGGAATCCTGGTGGAATACATTTAATGAGAAGTGGG
(SEQ ID NO: 7)
ACTTCTCATTAAATGTATTCCACCAGGATTCCCATTCATC
(SEQ ID NO: 8)
CCCACTTCTCATTAAATGTATTCCACCAGGATTCCCATTCATC

An exemplary target sequence in TM3 includes SEQ ID NO: 9, or its complement on the antiparallel strand, SEQ ID NO: 10. The PAM sequence in each strand can also be included in the target sequence, so that the target sequences can include SEQ ID NO: 11 or its complement SEQ ID NO: 12. A gRNA complementary to TM3, designated m3, can therefore include a spacer sequence complimentary to SEQ ID NO: 9, SEQ ID NO: 10, SEQ ID NO: 11; or SEQ ID NO: 12.

The nucleotide sequences are as follows:

(SEQ ID NO: 9)
AAGGTACTGGCTATTCAAGGGGCCAATAGACAG
(SEQ ID NO: 10)
AAGGTACTGGCTATTCAAGGGGCCAATAGACAGTGG
(SEQ IN NO: 11)
CTGTCTATTGGCCCCTTGAATAGCCAGTACCTT
(SEQ ID NO: 12)
CCACTGTCTATTGGCCCCTTGAATAGCCAGTACCTT

Stretches of DNA containing the target sites for TM1, TM2, and TM3 are diagramed in FIG. 1A, and their nucleotide sequences are shown in FIG. 1B as SEQ ID NOS: 13-18. It will be understood that the gRNAs of the present invention can also include additional 5′ and/or 3′ sequences that may or may not be complementaryto a target sequence. The spacers of each gRNA can have less than 100% complementarity to its target sequence, for example 95% complementarity.

In Examples 2, 4, and 5, CRISPR systems including Cas9 and gRNAs m1, m2, and/or m3 were found to inhibit JCV replication and T-Ag expression in host cells, and to damage the integrity of the JCV genome. These effects cause the elimination of both free episomal virus, and virus integrated into host genomes. Harmful off-target effects on healthy genes were not produced. Therefore, the present invention encompasses a composition for use in eliminating JCV from a host cell infected with JCV. The composition includes at least one isolated nucleic acid sequence encoding a CRISPR-associated endonuclease, and at least one gRNA having a spacer sequence complementary to a target sequence in a JCV DNA. The present invention also encompasses a method of eliminating JCV from a host cell infected with JCV. The method includes the steps of treating the host cell with a composition comprising a CRISPR-associated endonuclease, and at least one gRNA having a spacer sequence that is complementary to a target sequence in a JCV DNA; and eliminating the JCV from the host cell.

In the experiments of Example 3, it was determined that stable expression of the CRISPR systems of the present invention can render host cells refractory to new infection by JCV. Therefore, the present invention also encompasses a method of preventing JCV infection of cells of a patient at risk of JCV infection. The method includes the steps of determining that a patient is at risk of JCV infection; exposing cells of the patient at risk of JCV infection to an effective amount of an expression vector composition that includes an isolated nucleic acid encoding a CRISPR-associated endonuclease, and at least one isolated nucleic acid encoding at least one guide gRNA including a spacer sequence complementary to a target sequence in a JCV DNA; stably expressing the CRISPR-associated endonuclease and the at least one gRNA in the cells of the patient; and preventing JCV infection of the cells of the patient.

The gRNAs of the present invention can be configured as a single sequence or as a combination of one or more different sequences, e.g., a multiplex configuration. Multiplex configurations can include combinations of two, three, or more different gRNAs. When the compositions are administered in an expression vector, the guide RNAs can be encoded by a single vector. Alternatively, multiple vectors can be engineered to each include two or more different guide RNAs. Especially useful care combinations of gRNAs that cause the excision of viral sequences between cleavage sites, resulting in the ablation of the JCV genome orJCV protein expression. The excised region can vary in size from a single nucleotide to several hundred nucleotides.

The RNA molecules (e.g., crRNA, tracrRNA, gRNA) may be engineered to comprise one or more modified nucleobases. For example, known modifications of RNA molecules can be found, for example, in Genes VI, Chapter 9 (“Interpreting the Genetic Code”), Lewin, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2thiouridine; 2-thio-2′-O-methyluridine; 3,2′-O-dimethyluridine; 3-(3-amino-carboxypropyl)uridine; 4-thiouridine; ribosylthymine; 5,2′-O-dimethyluridine; 5-methyl-2thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5methoxycarbonylmethyl-2′-O-methyluridine; 5-methoxycarbonylmethyl-2′-thiouridine; 5-carbamoyl methyluridine; 5-carba moylmethyl-2′-O-methylu rid ine; 5-(carboxyhydroxymethyl) uridine; 5-(carboxyhydroxymethyl) uridinemethyl ester; 5-aminomethyl-2-thiouridine; 5methylaminomethyluridine; 5-methylaminomethyl-2-thiouridine; 5-methylaminomethyl-2selenouridine; 5-carboxymethylaminomethyluridine; 5-carboxymethylaminomethyl-2′-Omethyl-uridine; 5-carboxymethylaminomethyl-2-thiouridine; dihydrouridine; dihydroribosylthymine; 2′-methyladenosine; 2-methyladenosine; N⁶-methyladenosine; N⁶,N⁶-dimethyladenosine; N⁶,2′-O-trimethyladenosine; 2-methylthio-N⁶ N-isopentenyladenosine; N⁶-(cis-hydroxyisopentenyI)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶threonylcarbamoyl adenosine; 2-methylthio-N⁶-methyl-N⁶-threonylcarbamoyl adenosine; N⁶hydroxynorvalylcarbamoyl adenosine; 2-methylthio-N⁶-hydroxnorvalylcarbamoyl adenosine; 2-O-ribosyladenosine (phosphate); inosine; 2′O-methyl inosine; 1-methyl inosine; 1;2′-O-dimethy linosine; 2′-O-methyl guanosine; 1-methyl guanosine; N²-methyl guanosine; N2,N2-dimethyl guanosine; N2,2′-O-dimethyl guanosine; N²,N²,2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; 30 hydroxywybutosine; peroxywybutosine; queuosine; epoxyqueuosine; galactosyl-queuosine; mannosyl-queuosine; 7-cyano-7-deazaguanosine; arachaeosine [also called 7-formarnido-7-deazaguanosine]; and 7-aminomethyl-7-deazaguanosine. The methods of the present invention or others in the art can be used to identify additional modified RNA molecules.

The gRNAs of the present invention are not limited to those complementary to sequences found within the TM1, TM2 or TM3 region of JCV T-antigen. Other regions of JCV, and other polyomaviruses, can be targeted by CRISPR systems with suitably designed gRNAs. For CRISPR systems employing S. pyogenes Cas9, the PAM sequence can be AGG, TGG, CGG or GGG. Candidate target sequences can be identified by proximity to a 5′ PAM such as AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the gRNA may vary, but useful gRNA sequences will be those that minimize off target effects while achieving high efficiency and complete elimination of JCV. Efficiency and off target effects of candidate gRNAs can be determined by the assays disclosed in Examples 1-5.

The present invention is not limited to Cas9 endonucleases. It also encompasses compositions and methods entailing the use of any CRISPR associated endonuclease that is capable of cleaving a viral genome after guidance to a PAM site by a gRNA. Examples include endonucleases of the family Cpf1 (CRISPR from Prevotella and Francisella 1) (Zetsche, et aL, 2015). Two Cpf1 endonucleases have so far been shown to be effective at editing genes in a cultured human kidney cell system: Acidaminococcus sp. BV3L6 Cpf1, and Lachnospiraceae bacterium ND2006 Cpf1.

Cpf1 endonucleases expand the range of possible targets in JCV and other polyomaviruses, because they recognize a PAM different from the cytosine rich PAM recognized by Cas9. Cpf1 recognizes a thymine rich PAM, with a consensus sequence TTN, and that PAM is located at the 5′ end of the target sequence. Cpf1 is guided by a smaller, simpler gRNA than that of Cas9 systems. Instead of a two-unit gRNA including crRNA and tracrRNA, or an engineered chimeric hybrid of crRNA and tracrRNA, Cpf1 is guided by single guide RNA, termed gRNA. The Cpf1 molecule is also smaller than the Cas9 molecule. This greater simplicity and smaller size facilitates both the design and use of CRISPR/Cpf1 systems, and the delivery of the endonuclease component to the nucleus of a host cell.

The sequences of gRNAs will depend on the sequence of specific target sites selected for editing. The preferred target sites are situated in the T-Ag region of JCV or another polyomavirus. In general, the gRNAs are predicted to be complementary to target DNA sequences that are immediately 3′ to a thymine rich PAM, of sequence 5′TTN. The gRNA sequence can be complimentary to a sense or anti-sense sequence. The gRNA sequence may or may, not include the complement to the PAM sequence. The gRNA sequence can include additional 5′ and/or 3′ sequences that may not be complementary to a target sequence. The gRNA sequence can have less than 100% complementarity to a target sequence, for example 95% complementarity. The gRNA have a sequence complimentary to a coding or a non-coding target sequence. The gRNA sequences can be employed in a multiplex configuration, including combinations of two, three, four, five, six, seven, eight, nine, ten, or more different gRNAs.

The compositions and methods of the present invention have proven effective in eliminating JCV, by gRNA guided attack on the T-Ag gene. It is therefore likely that the present invention is readily adaptable to serve as an effective treatment for other polyomaviruses, such as SV40. Adaptation is mainly a matter of identifying PAM sequences for Cas9, or another suitable endonuclease, in the T-Ag gene or other critical genes, of the particular polyomavirus. gRNAs complimentary to sequences adjacent to the PAM sequences are then generated and tested by the methodology disclosed in Examples 1-5.

Therefore, the present invention encompasses a method of eliminating a polyomavirus from a host cell infected with a polyomavirus. The method includes the steps of treating the host cell with a CRISPR associated endonuclease, and at least one guide gRNA having a spacer sequence that is complementary to a target sequence in a polyomavirus DNA; and eliminating the polyomavirus from the host cell. In preferred embodiments, gRNA spacer sequences are complementary to target sequences in the large T-antigen (T-Ag) encoding region of the polyomavirus DNA.

Vectors. The present invention includes a vector comprising one or more casettes for expression of CRISPR components such as one or more gRNAs and a Cas endonuclease such as Cas9. The vector can be any vector that is known in the art and is suitable for expressing the desired expression cassette. A number of vectors are known to be capable of mediating transfer of gene products to mammalian cells, as is known in the art and described herein. A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy.

A preferred vector is a lentiviral vector. Lentiviral vectors have the advantage of providing efficient transduction of both proliferating and resting cells, stable expression of delivered genes by integration into host chromatin, and the absence of interference from preexisting viral immunity. In experiments disclosed in Example 5, drug-inducible lentiviral expression vectors for Cas9/gRNA components were shown to be effective in ablating JCV T-Ag expression in infected cells. In an exemplary configuration, host cells were stably transduced with Cas9 or another suitable CRISPR endonuclease in doxycycline inducible lentiviral vector. When elimination of JCV was desired, the host cells were transduced with one or more gRNAs and treated with doxycycline, to activate expression of Cas9, to cause guided cleavage of the JCV genome and inactivation of virus. Alternatively, one or more gRNAs can be transduced stably, in a drug-inducible manner, or both a CRISPR associated endonuclease and gRNAs can be so transduced. In a clinical situation, this treatment could be used for patients at risk of JCV infection, with the CRISPR components being activated upon infection.

Therefore, the present invention encompasses a vector composition for use in eliminating JCV from a host cell. The vector composition includes at least one isolated nucleic acid sequence encoding a CRISPR-associated endonuclease, and at least one gRNA having a spacer sequence complementary to a target sequence in a JCV DNA. The isolated nucleic acid sequences are included in at least one expression vector, which induces the expression of the CRISPR-associated endonuclease and the at least one gRNA in a host cell.

The present invention is by no means limited to the plasmid and lentiviral vectors described in Examples 1-5. Other preferred vectors include adenovirus vectors and adeno-associated virus vectors. These have the advantage of not integrating into host cell DNA. Many other recombinant viral vectors are also suitable, including, but not limited to, vesicular stomatitis virus (VSV) vectors, pox virus vectors, and retroviral vectors.

A “recombinant viral vector” refers to a viral vector comprising one or more heterologous gene products or sequences. Since many viral vectors exhibit size constraints associated with packaging, the heterologous gene products or sequences are typically introduced by replacing one or more portions of the viral genome. Such viruses may become replication defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described.

Retroviral vectors include Moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. In some embodiments, a high expression promoter can be used. An example of a suitable promoter is the 763-base-pair cytomegalovirus (CMV) promoter. The Rous sarcoma virus (RSV) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

Another delivery method is to use single stranded DNA producing vectors which can produce the expressed products intracellularly. See for example, Chen et al, BioTechniques, 34: 167-171 (2003), which is incorporated herein, by reference, in its entirety.

Expression may be controlled by any promoter/enhancer element known in the art that is functional in the host selected for expression. Besides the promoters described in the examples section, other promoters which may be used for gene expression include, but are not limited to, cytomegalovirus (CMV) promoter, the SV40 early promoter region, the promoter contained in the 3′ long terminal repeat of Rous sarcoma virus, the herpes thymidine kinase promoter, the regulatory sequences of the metallothionein gene; prokaryotic expression vectors such as the beta-lactamase, or the tac promoter; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus.

A wide variety of host/expression vector combinations may be employed in expressing the nucleic acid sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Yeast expression systems can also be used. For example, the non-fusion pYES2 vector (XbaI, SphI, ShoI, NotI, GstXI, EcoRI, BstXI, BamH1, SacI, KpnI, and HindIII, cloning sites; Invitrogen) or the fusion pYESHisA, B, C (XbaI, SphI, ShoI, NotI, BstXI, EcoRI, BamH1, SacI, KpnI, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

Additional suitable vectors include v fusion proteins and chemical conjugates. If desired, the polynucleotides of the invention may also be used with a microdelivery vehicle such as cationic liposomes and other lipid-containing complexes, and other macromolecular complexes capable of mediating delivery of a polynucleotide to a host cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components can also include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al.; BioTechniques, 534: 167-171 (2003). A large variety of such vectors are known in the art and are generally available.

Pharmaceutical Compositions

The compositions and methods that have proven effective for elimination of JCV from cultured cells (Examples 1-5) are very likely to be effective in vivo, if delivered by means of one or more suitable expression vectors. Therefore, the present invention encompasses a pharmaceutical composition for the elimination of JCV from the cells of a mammalian subject, including an isolated nucleic acid sequence encoding a CRISPR-associated endonuclease, and at least one isolated nucleic acid sequence encoding at least one gRNA that is complementary to a target sequence in the JCV genome. The preferred gRNAs include spacer sequences that are complementary to the TM1, TM2, TM3, or other regions of the JCV T-Ag. For example, gRNAs m1, m2, and m3 can be included, individually or in any combination. It is also preferable that the pharmaceutical composition also include at least one expression vector in which the isolated nucleic acid sequences are encoded.

Pharmaceutical compositions according to the present invention can be prepared in a variety of ways known to one of ordinary skill in the art. For example, the nucleic acids and vectors described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein, in combination with one or more pharmaceutically acceptable carriers. We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid. Further description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μrn in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are common latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating a nucleic acid encoding Cas9 or a variant Cas9 and at least one gRNA sequence complementary to a target HIV; or it can include a vector encoding these components. Alternatively, the compositions can be formulated as a nanoparticle encapsulating the CRISPR-associated endonuclease the polypeptides encoded by one or more of the nucleic acid compositions of the present invention.

Methods of Treatment

The results disclosed in Examples 1-4 show that the CRISPR systems of the present invention are effective at eliminating JCV from host cells, and at rendering host cells refractory to new infection. These findings indicate that the components of the CRISPR systems can be delivered to patients infected with JCV, and those at risk of JCV, as curative and prophylactic treatments. Preferred methods of delivery include the previously described pharmaceutical compositions.

The present invention therefore encompasses a method of treating a subject having a LCV related disorder, such as progressive multifocal leukoencephalopathy, including the step of administering, to the subject, an effective amount of the previously described pharmaceutical composition. The present invention also encompasses a method of preventing JCV infection of cells of a patient at risk of JCV infection, including the steps of: determining that a patient is at risk of JCV infection; exposing cells of the patient to an effective amount of an expression vector composition including an isolated nucleic acid encoding a CRISPR-associated endonuclease, and at least one isolated nucleic acid encoding at least gRNA including a spacer sequence complementary to a target sequence in a JCV DNA; stably expressing the CRISPR-associated endonuclease and the at least one gRNA in the cells of the patient; and preventing JCV infection of the patient's cells.

The compositions and methods of the invention are generally and variously useful for treatment of a subject having a polyomavirus-mediated infection, for example, a JCV infection. A subject is effectively treated whenever a clinically beneficial result ensues. This may mean, for example a complete resolution of the symptoms of a disease, a decrease in the severity of the symptoms of the disease, or the slowing of the disease's progression. Thus, the methods of the invention may be useful for treatment of diseases and disorders associated with JCV infections, such as adrenal neuroblastoma, neuroectodermal tumors, tumors originating from the cerebellum, pituitary neoplasia, peripheral nerve sheath tumors, and cancer, including brain cancers such as oligoastrocytoma, xanthoastrocytoma, medulloblastomas, oligodendroglioma, glioblastoma multiforme, brain tumors of glial origin, CNS lymphoma; as well as cancers of the colon and esophagus; or secondary infections. The methods can further include the steps of a) identifying a subject (e.g., a patient and more specifically, a human patient) who has a JCV infection and b) providing to the subject a composition comprising a nucleic acid encoding a CRISPR-associated endonuclease and a guide RNA complementary to a JCV target sequence, for example, a T-antigen sequence. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as to predict outcome.

In some methods of the present invention, one can first determine whether a patient has a JCV infection, and then make a determination as to whether or not to treat the patient with one or more of the compositions described herein. Monitoring can also be used to detect the onset of drug resistance and to rapidly distinguish responsive patients from nonresponsive patients. In some embodiments, the methods can further include the step of determining the nucleic acid sequence of the particular JCV harbored by the patient and then designing the guide RNA to be complementary to those particular sequences. For example, one can determine the nucleic acid sequence of a subject's TM1, TM2 and/or TM3 region and then design one or more gRNAs to be precisely complementary to the patient's sequences.

In some embodiments, the compositions can be administered ex vivo to treat cells or organs removed from one individual for transplantation into another individual. Cells or organs intended for transplantation can be transduced with a vector comprising the Cas9 compositions of the invention. Removal or attenuation of the target JCV sequences can be confirmed, and the treated cells infused into the patient.

The compositions or agents identified by the methods embodied herein may be administered to subjects including animals and human beings in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice.

The compositions of the invention may be administered to a subject by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, useful compositions are formulated in a sterilized pyrogen-free form.

The compositions can be administered with an additional therapeutic agent, for example, an agent used for the treatment of a demyelinating disease, e.g, Natalizumab (Tysabri®), Fingolimod (Gilenya); B cell dysfunction (Rituximab (Rituxan®, mabThera® and Zytux®); immune-mediated disorders and transplantation (Adalimumab/Humira; Brentuximab vedotin/Adcetris; Efalizumab/Raptiva; Etanercept/Enbrel, Infliximab (Remicade); Mycophenolate mofetil (MMF)/Cell Cept; or a retroviral infection, e.g., HAART (Highly Effective Antiretroviral Therapy). Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen, e.g., continuous low-doses of a therapeutic agent.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The Cas9/gRNA compositions that exhibit high therapeutic indices are preferred. While Cas9/gRNA compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions lies generally within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

The compositions can include a cell which has been transformed or transfected with one or more Cas/gRNA vectors. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell, for example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are a self-derived cell, make it possible to avoid rejection reactions, which are the biggest obstacle to regenerative medicine or transplantation therapy.

The gRNA expression cassette can be delivered to a subject by methods known in the art. In some aspects, the Cas may be a fragment wherein the active domains of the Cas molecule are included, thereby cutting down on the size of the molecule. Thus, the Cas9/gRNA molecules can be used clinically, similar to the approaches taken by current gene therapy. In particular, a Cas9/multiplex gRNA stable expression stem cell or iPS cells for cell transplantation therapy as well as polyomavirus vaccination will be developed for use in subjects.

Transduced cells are prepared for reinfusion according to established methods. After a period of about 2-4 weeks in culture, the cells may number between 1×10⁶ and 1×10¹° . In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are nₒₜ restricted to, bone marrow or adipose tissues.

### Articles of Manufacture

The compositions described herein can be packaged in suitable containers labeled, for example, for use as a therapy to treat a subject having a retroviral infection, for example, a JCV infection or a subject at risk for a JCV infection. The containers can include a composition comprising a nucleic acid sequence encoding a CRISPR-associated endonuclease, for example, a Cas9 endonuclease, and a guide RNA complementary to a target sequence in a JCV, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in a JCV, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

In some embodiments, the kits can include one or more additional therapeutic agents as described above. The additional agents can be packaged together in the same container as a nucleic acid sequence encoding a CRISPR-associated endonuclease, for example, a Cas9 endonuclease, and a guide RNA complementary to a target sequence in a JC virus, or a vector encoding that nucleic acid or they can be packaged separately. The nucleic acid sequence encoding a CRISPR-associated endonuclease, for example, a Cas9 endonuclease, and a guide RNA complementary to a target sequence in a JC virus, or a vector encoding that nucleic acid and the additional agent may be combined just before use or administered separately.

The product may also include a legend (e.g., a printed label or insert or other medium describing the product's use (e.g., an audio- or videotape). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

### EXAMPLES

### Example 1

### Materials and Methods

The effects of Cas9 and gRNAs in targeting T-antigen and its function were examined using the CRISPR/Cas9 technology. Design of guide RNAs for CRISPR/Cas9 targeting of JCV is shown in FIGS. 1A and 1B.

Cell culture. The human oligodendroglioma cell line TC620 (Wollebo, et al., 2011) and SVG-A, a cell line derived from primary human fetal glial cells transformed by origin-defective SV40 that expresses SV40 T-Ag (Major, et al., 1985), were maintained in Dulbecco's Modified Eagle's Medium (DMEM) supplemented with 10% fetal bovine serum (FBS) as previously described (Wollebo, et al., 2011). HJC-2 is a JCV-induced hamster glioblastoma cell line that expresses JCV T-Ag (Raj, et al., 1995). BsB8 is a mouse cell line derived from a tumor of cerebellar neuroectodermal origin arising in transgenic mice expressing the JCV early protein T-Ag (Krynska, et al., 2000). Derivatives of SVG-A cells expressing Cas9 and JCV T antigen gRNAs were developed by transfection of SVG-A cells with the pX260-derived plasmids (described below), selection in puromycin containing media, and isolation of single clones by dilution cloning.

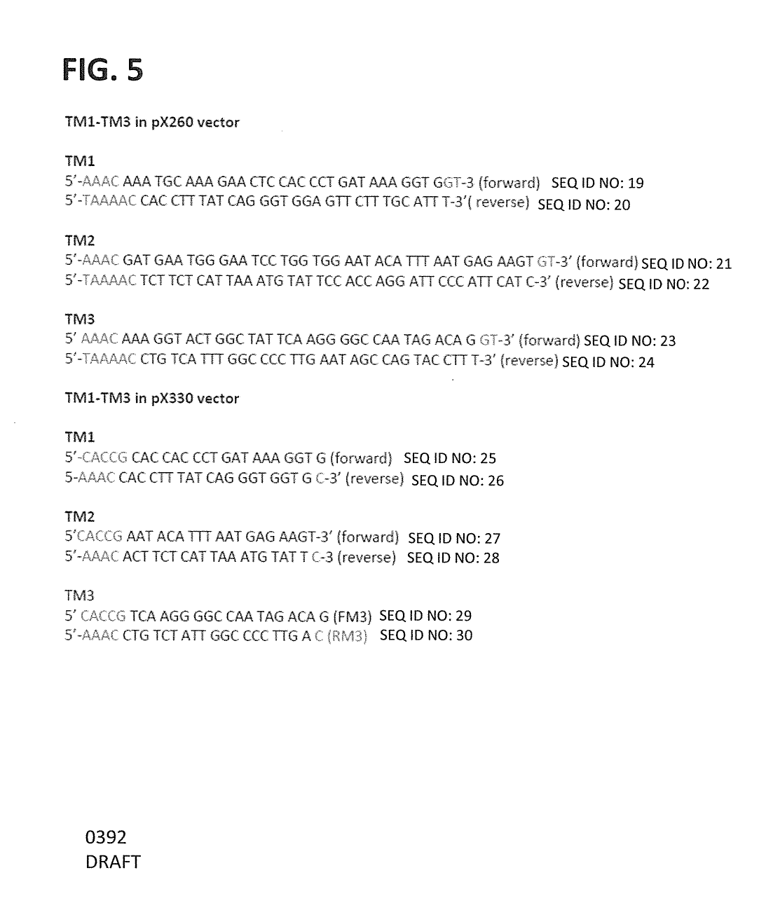

Plasmid preparation. Vectors containing the human Cas9 and gRNA expression cassette, pX260 and pX330 (Addgene) were used to create various constructs. Both vectors contain a humanized Cas9 coding sequence driven by a CAG promoter and a gRNA expression cassette driven by a human U6 promoter (Cong, et al., 2013). The vectors were digested with Bbsl and treated with Antarctic Phosphatase. A pair of oligonucleotides for each targeting site was annealed, phosphorylated, and ligated to the linearized vector. The oligonucleotides are shown in FIG. 5 (SEQ ID NOS: 19-30). The gRNA expression cassette was sequenced with a U6 sequencing primer in GENEWIZ. For pX330 vectors, a pair of universal PCR primers was designed with overhang digestion sites that can tease out the gRNA expression cassette (U6-gRNA-crRNA-stem-tracrRNA) for direct transfection or subcloning to other vectors.

The reporter construct, JCV_{L}-LUC (Wollebo, et al, 2011) and the pcDNA 3.1-large T-Ag expression plasmid have been described previously (Chang, et al., 1996). For lentivirus production, the following plasmids were obtained from Addgene: pCW-Cas9 (#50661), psPAX2 (#12260), pCMV-VSV-G (#8454), pKLV-U6gRNA(Bbsl)-PGKpuro2ABFP (#50946), pMDLg/pRRE (#12251), pRSV-Rev (#12253). To construct the gRNA lentiviral expression plasmids for each of the three targets, the U6 expression cassette from each of the three pX330 gRNA plasmids described above was amplified by PCR with flanking primers (5′-tatgggcccacgcgtgagggcctatttcccatgattcc-3′ (SEQ ID NO: 42) and 5′-tgtggatcctcgaggcgggccatttaccgtaagttatg-3′) (SEQ ID NO: 43) and the PCR products treated with MluI and BamHI and cloned into pKLV-U6gRNA(BbsI)-PGKpuro2ABFP that had been cut with MluI and BamHI. The pCR 4-TOPO TA vector was from Life Technologies, Inc., Carlsbad, Calif.

Transient transfection and reporter assays. Co-transfection of JCV_{L}-LUC reporter plasmid and T-Ag expression plasmid was performedas previously described (Wollebo, et al., 2011, Wollebo, etal., 2012). Briefly, TC620 cells were transfected with either reporter constructs alone (200 ng) or in combination with the various expression plasmids for 48 h prior to harvesting. The total amount of transfected DNA was normalized with empty vector DNA. Luciferase assay was performed as previously described (Wollebo, et al., 2011, Wollebo, etal., 2012).

Production of clonal derivatives of SVG-A expressing Cas9 and gRNAs. SVG-A cells were transfected with pX260 or pX260-derived plasmids expressing each of the three previously described gRNAse. Selection was done with 3 μg/ml puromycin and clones isolated by dilution cloning.

Assay of JCV infection. Infection experiments were performed with SVG-A cells or the SVG-A clonal derivatives of SVG-A expressing Cas9 and gRNAs described above. Cells were infected with Mad-1 JCV at an MOI of 1 as previously described (Radhakrishnan, et al., 2003 Radhakrishnan, et al., 2004); and harvested and analyzed after 7 days together with uninfected control cultures. Expression of the viral proteins VP1 and agnoprotein was measured in whole cell protein extracts by Western blot. In parallel, the growth media of the cells was also collected to measure viral load by Q-PCR.

Immunocytochemistry. TC620 cells were transfected with an expression plasmid for FLAG-tagged Cas9, and immunocytochemistry was performed with mouse anti-Flag M2 primary antibody (1:500, Sigma) as previously described (Hu, et al., 2014).

Analysis of T-Ag gene cleavage by PCR. BsB8 or HJC-2 cells were transfected with plasmid pX260 or pX260-derived plasmids expressing the gRNAs described above. Total genomic DNA was extracted after 48 hours. The T-Ag gene was amplified by PCR using primers that flank the JCV T-Ag coding region (5′-gcttatgccatgccctgaaggt-3′ (SEQ ID NO: 44) and 5′-atggacaaagtgctgaataggga-3′ (SEQ ID NO: 45) and PCR products were subjected to agarose gel electrophoresis.

Production of lentiviral vectors for Cas9 and creation of HJC-2 cells expressing inducible Cas9. To produce a lentiviral vector for transduction of doxycycline-inducible Cas9 expression, 293T cells were transfected with plasmids pCW-Cas9, psPAX2, and pCMV-VSV-Gusing by the calcium phosphate precipitation method (Graham and van der Eb, 1973). Lentivirus was harvested from the supernatant after 48 h, cleared by centrifugation, and passed through a 0.45 μm filter as previously described (Wollebo, et al., 2013). To obtain stably transduced HJC-2 clonal cell derivatives inducibly expressing Cas9, lentivirus was added to HJC-2 cells in the presence of 6 μg/ml polybrene, followed by selection with 3 μg/ml puromycin and isolation of clones by dilution cloning. For induction of Cas9 expression in the resulting clones, 2 μg/ml doxycycline was added to the culture media.

Production of lentiviral vectors for gRNAs and transduction of HJC-2 cells expressing inducible Cas9. To produce a lentiviral vector for transduction of the three gRNAs, each of the three gRNA lentiviral expression plasmid derivatives, constructed as described above from pKLV-U6gRNA (Bbs1)-PGKpuro2A8FP, were transfected into 293T cells by calcium chloride precipitation together with packaging plasmids pCMV-VSV-G, pMDLg/pRRE and pRSV-Rev. Lentivirus was harvested from the supernatant after 48 h, cleared by centrifugation, passed through a 0.45 μim filter, and added to HJC-2 cells in the presence of 6 μg/m1polybrene followed by selection. After 24 hours, the transduced cells were treated with 2 μg/ml doxycycline to induce Cas9 expression and after another 48 hours harvested and analyzed for T-Ag mutations by PCR/sequencing, for T-Ag expression by Western blot, and for clonogenicity by colony formation assay.

Analysis of InDel mutations. Since the cleavage of DNA by Cas9 leaves behind characteristic short insertion/deletion (InDel) mutations, The sequence of the T-Ag gene from the gRNA-transduced HJC-2 cells was analyzed for InDels. Total genomic DNA was isolated from cells using a genomic DNA purification kit according to the manufacturer's instructions (5prime Inc., Gaithersburg Md.), and the regions of the T-Ag gene that had been targeted were amplified by PCR using flanking primers. For TM1 and TM2, the following primers used were 5′-ctctggtcatgtggatgctgt-3′ (SEQ ID NO: 46) and 5′-atggacaaagtgctgaataggga-3′ (SEQ ID NO: 47). Primers 5′-gcttatgccatgccctgaaggt-3′ (SEQ ID NO: 48) and 5′-acagcatccacatgaccagag-3′ (SEQ ID NO: 49) were used for TM3. The PCR products were cloned into the TA cloning vector pCR4-TOPO and colonies sequenced.

SURVEYOR assay. The presence of mutations in PCR products derived from HJC-2 cells expressing Cas9, and transduced by lentiviral vectors for gRNAs, was examined using the SURVEYOR Mutation Detection Kit (Transgenomic) according to the manufacturer's protocol. The same primers were used as described for the InDel mutation analysis. The heterogeneous PCR product was denatured for 10 min at 95° C. and then hybridized by gradual cooling using a thermocycler. Three hundred nanograms of hybridized DNA (9 μl) was digested with 0.25 μl of SURVEYOR Nuclease, which is a mismatch-specific DNA endonuclease used to scan for mutations in heteroduplex DNA; plus 0.25 μl SURVEYOR Enhancer S and 15 mM MgCl2 for 4 h at 42° C. Stop Solution was added, and samples were resolved on a 2% agarose gel, together with equal amounts of control samples. The control samples were treated in parallel, but were derived from HJC-2 cells expressing Cas9 but not transduced by lentiviral vector for gRNA. The SURVEYOR assay was also used to detect the presence of off-target mutations on cellular genes using stable derivatives of SVG-A cell lines expressing Cas9 and gRNAs.

Colony formation assay. HJC-2 cells expressing inducible Cas9 were transduced with lentiviral expression vectors for gRNAs for each of the three targets, either alone or in combination. Cells were plated for colony formation assays in the presence or absence of doxycycline. Cells were grown for 10-14 days, washed with PBS, and fixed and stained with 40% methanol and 0.4% methylene blue. Colonies with more than 50 cells were counted.

### Example 2

### Expression of gRNAs Targeting the T-Ag Reduces Both T-Ag Expression and T-Ag Stimulated JCV Late Gene Expression in TC620 Cells Transfected with T-Ag and Cas9

In a first set of experiments, it was determined whether Cas9, in combination with the various gRNAs targeting TM1, TM2 and TM3, can suppress expression of T-antigen in the human oligodendrocytic cell line, TC620. In these experiments, gRNA m1, is complementary to the TM1 target sequence SEQ ID NO: 1; gRNA m2, is complementary to the TM2 target sequence SEQ ID NO: 5; and gRNA m3, is complimentary to the TM3 target sequence SEQ ID NO: 9.

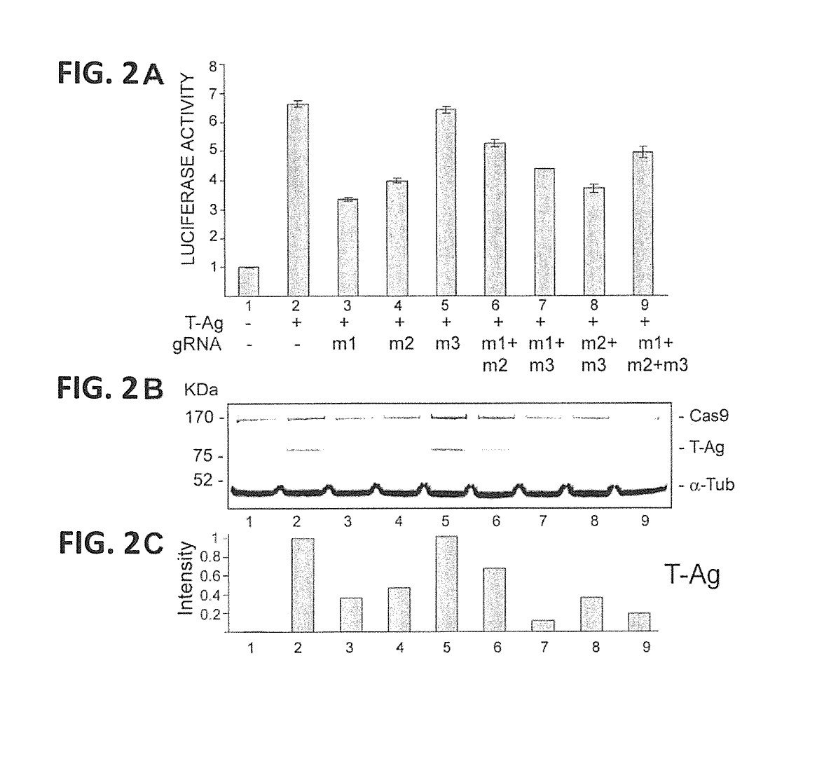

Results from Western blot analysis of TC620 cells transfected with plasmid expressing T-antigen, either alone or in combination with Cas9, and/or in combination with T-Ag targeting gRNA expression plasmids, are shown in FIG. 2A. As seen in this figure, the presence of Cas9 together with either m1 or m2 gRNAs noticeably decreased the level of T-Ag production in the transfected cells. However, expression of gRNA m3 showed no significant effect on the level of T-Ag expression. FIG. 2B show results from quantification of T. antigen production based on the intensity of the band corresponding to T-antigen, which was normalized to the housekeeping gene, β-tubulin.

Next, functional studies were performed, to gauge the ability of T-Ag to stimulate JCV late promoter activity (Lashgari, et al., 1989]. TC620 cells are particularly appropriate in this study, as their oligodendrocytic origin allows cell type-specific transcription of the JCV promoter at the basal level, which can be induced upon T-Ag expression. Results from transcription studies using the JCV late promoter, JCV_{L}, driving the reporter luciferase gene, showed that the level of JCV_{L} promoter activation by T-Ag is significantly decreased in cells expressing Cas9/gRNA m1 or Cas9/gRNA m2, but not Cas9/gRNA m3 (FIG. 2C). Taken together, these observations show that gRNAs m1 and m2, alone or in combination, when expressed together with Cas9, reduce T-Ag expression and inhibit subsequent T-Ag-stimulated events related to viral lytic infection. The outcome of these effects is the elimination of JCV from host cells.

### Example 3

### A Clonal Derivative of SVGA Expressing Cas9 and gRNA Targeting the T-Ag has Reduced Capacity to Support JCV Infection

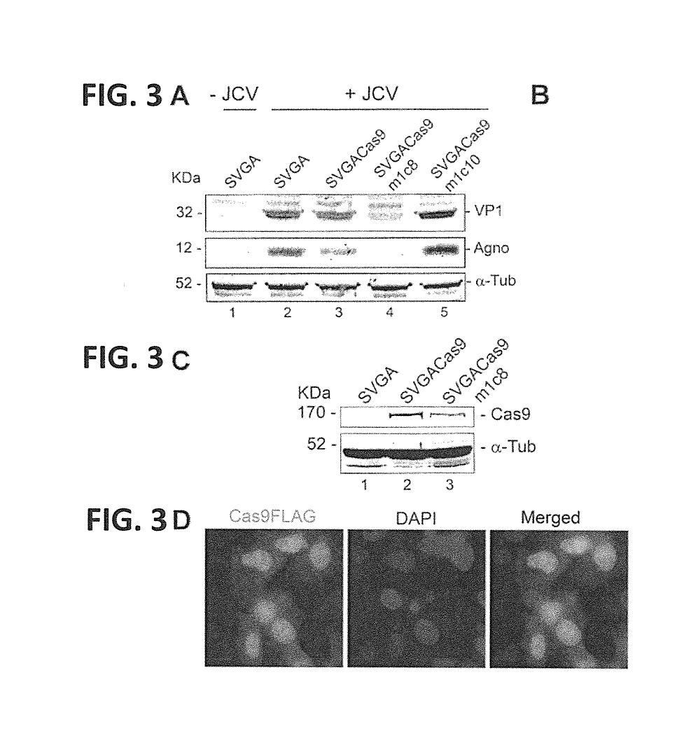

To investigate the effects of gRNA guided Cas9 upon JCV infection, experiments were performed with the SVG-A cell line. This line supports supports viral gene expression, and also allows for complete productive viral lytic infection. First, stable clonal cell lines were established from SVG-A cells that express either Cas9 or Cas9 plus gRNA ml. In these experiments, gRNA m1 was complementary to the TM1 target sequence SEQ ID NO: 1. Three separate clones were selected and used for JCV infection at an MOI=1.0 for seven days. Viral infection was assessed by Western blot analysis for the presence of the viral capsid protein, VP1 and the auxiliary protein, agnoprotein, with α-tubulin as a loading control. In parallel, quantitative PCR (Q-PCR) was carried out, to determine the level of viral DNA in the culture media, as a marker of DNA replication and therefore of virus production. As shown in FIG. 3A, the clonal cell line expressing only Cas9 (lane 3) supported JCV infection, as evidenced by the presence of the viral capsid protein, VP1 and the auxiliary agnoprotein in these cells (lane 2). In contrast, the SVG-A clone expressing both Cas9 plus gRNA m1 failed to support viral replication. This finding was supported by Q-PCR experiments to measure virus in the culture supernatants (FIG. 3B). Some of the clonal cells, which were originally transfected with both Cas9 and gRNA m1, were able to support JCV replication, suggesting that these cells either lost Cas9 expression and/or gRNA m1 production (data not shown). To determine whether JCV infection affects Cas9 expression, Cas9 expression was confirmed by Western blot in SVG-A Cas9 and SVG-A Cas9m1c8 cells (FIG. 3C) and FLAG-tagged Cas9 was shown by immunocytochemistry to localize to the nucleus (FIG. 3D).

The results show that stable expression of Cas9, and at least one gRNA targeting the JCV T-Ag gene, renders cells refractory to infection by JCV.

### Example 4

### Direct Demonstration of T-Ag Gene Cleavage After Transient Transfection of Cas9 and JCV T-Ag-Targeting gRNA

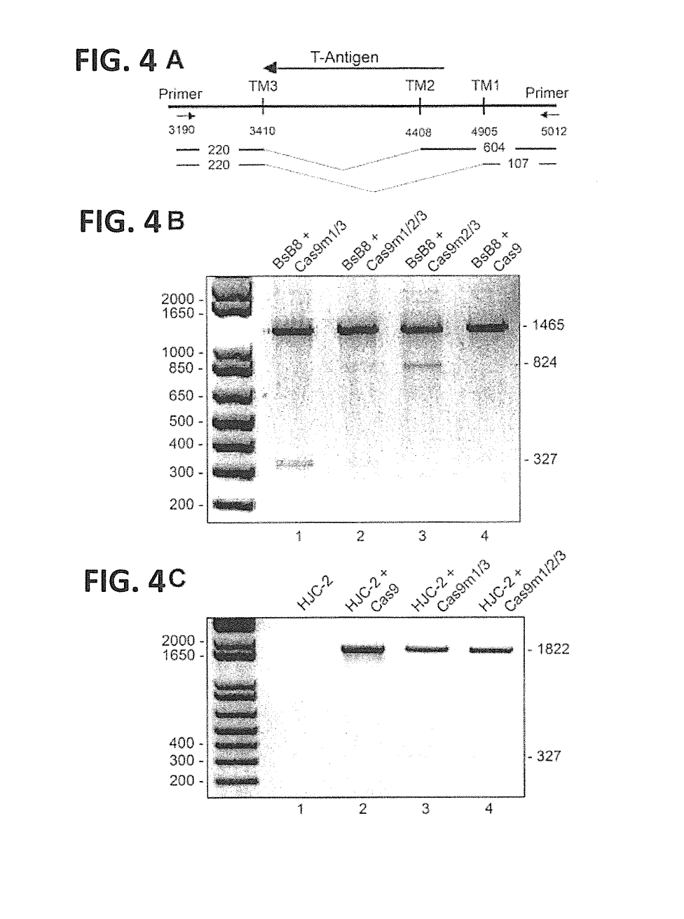

The ability of Cas9 and the gRNAs to edit the JCV DNA sequence corresponding to the T-Ag gene was next determined. For these experiments, BsB8 cells were utilized. BsB8 is a murine cell line that contains the JCV early region as an incorporated transgene, and that expresses T-Ag (Krynska, et al. 2000). Also employed was another cell line, HJC-2, a hamster cell line isolated by limiting dilution from a glioblastoma induced by intracerebral injection of JCV (Raj, et al., 1995). These cells also carry the JCV early genes integrated into the host genome, and express T-Ag. These cell lines were chosen for the experiments because their integrated copies of the JCV genome allow a precise measurement of the editing capabilities of the Cas9/gRNAs targeting the JCV sequences. The cells were transfected with expression plasmids for Cas9 and the gRNAs in various combinations, and genomic DNA was amplified using JCV-specific primers. FIG. 4A illustrates the positions of the cleavage points and the expected lengths of the resulting DNA fragments corresponding to the T-Ag gene after editing.

As shown in FIG. 4B, transfection of BsB8 cells with expression plasmids for Cas9 and gRNAs m1 and m3 (lane 1); m1, m2, and m3 (lane 2); and m2 and m3 (lane 3), resulted in the appearance of smaller fragments in addition to the expected intact 1465 base pair sequence (lane 4). As shown in FIG. 4C, transfection of HJC-2 cells with expression plasmids for Cas 9 and gRNAs m1 and m2, or m1, m2, and m3, also resulted in the appearance of cleavage products. FIGS. 4B and 4C show the appearance of a smaller fragment of 327 bp instead of the expected intact 1465 bp sequence, when a combination of gRNAs m1 and m3 were used to guide the cleavage of DNA. Further analysis suggested that the 327 bp fragment corresponds to the re-joining of the remaining DNA sequences (107+220) once the segment of the DNA between the target sequences m1 and m3 target sites is cleaved out. A similar event was observed when a multiplex of m2 and m3 was used in combination, leading to the genesis of an 824 bp DNA (604+220). These results indicate the cleavage of two gRNA-targeted regions within the viral genome, with the remaining gene sequences being re-joined by non-homologous end joining (NHEJ).

The results indicate that gRNAs according to the present invention, alone and in combination, induce cleavage and large deletions in JCV DNA, when they are expressed in combination with Cas9. This DNA damage can destroy the integrity of the JCV T-Ag gene and result in the elimination of virus from the host cells.

### Example 5

### Stable, Drug-Inducible Expression of Components of a Cas9/gRNA System Provides Drug-Inducible Ablation of T-Ag Expression in Host Cells

To determine whether JCV can be drug inducibly inactivated in a host cell, HJC-2 cells were transduced with a doxycycline inducible lentiviral vector encoding Cas 9. Stable derivatives were established. The derived cells were then transduced with lentiviral vectors encoding gRNAs specific for target sites in the coding region of the JCV T-Ag. The gRNAs included m1, m2, and m3 (with spacers complimentary to, respectively, SEQ ID NOS 1, 5, and 9. Following doxycycline induction, total genomic DNA was extracted. Regions of the T-Ag were amplified by PCR, cloned into TA vector and sequenced.

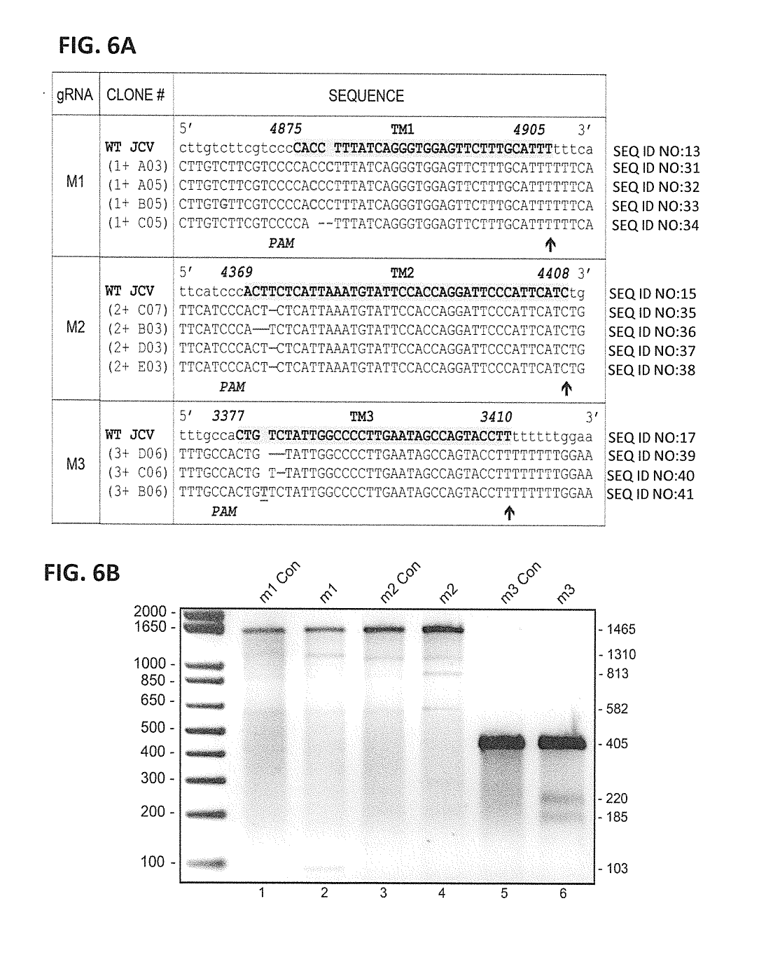

The Surveyor assay was used to detect InDel mutations in the PCR products (FIG. 6B). m1, m2, and m3 gRNAs all produced InDel mutations, as demonstrated by the presence of Surveyor nuclease cleavage products (FIG. 6B).

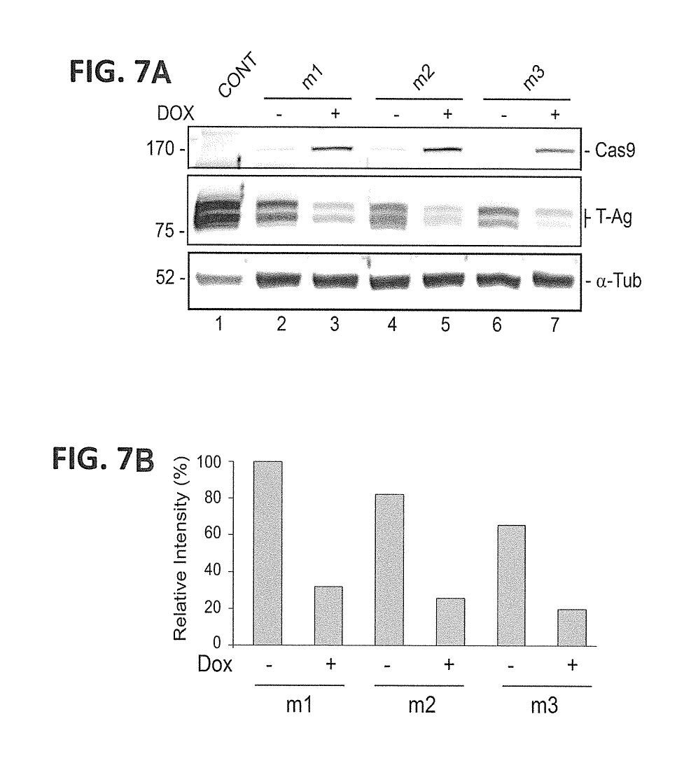

InDel mutations were detected in the corresponding region of the T-Ag gene of genomic DNA extracted from these cultures after 48 h, by sequencing of clones derived from PCR amplified products. Representative sequences including deletions or insertions are shown in FIG. 6A (SEQ ID NOS: 31-34, 35-36, and 39-41). The results show that most of the InDels were close to the PAM regions, and consisted of the insertion or deletion of one or two nucleotides. This would be predicted to cause frameshift mutations affecting T-Ag translation. As expected, it was determined by Western blot that T-Ag expression was ablated with each of the three gRNAs, upon the induction of Cas9 expression with doxycycline (FIG. 7A). FIG. 7B illustrates the densitometric quantitative analysis the Western blot results.

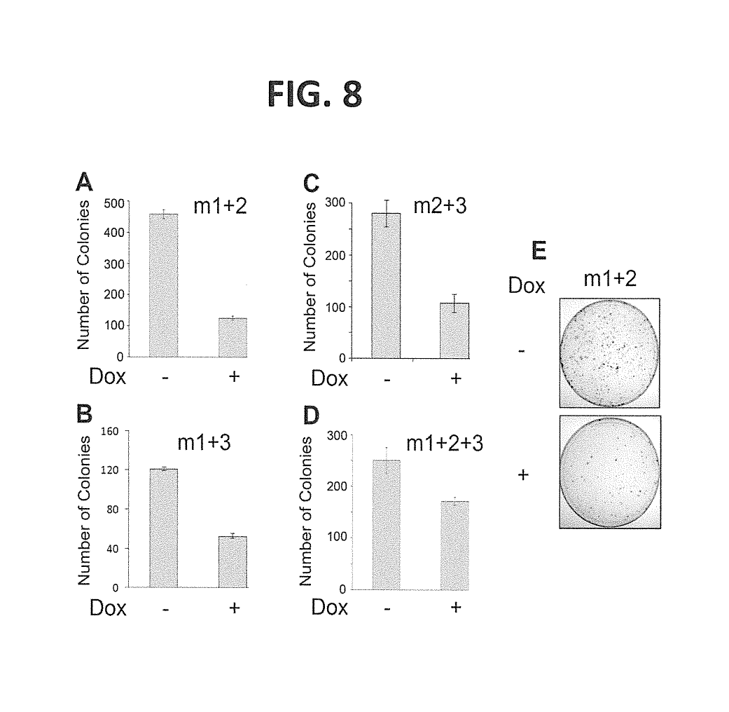

Also investigated was the effect of expression of Cas9 and gRNAS m1, m2 and m3, in various combinations, on the clonogenecity of HJC-2 cells. Clonogenicity in these cells is a phenotype that relies on the expression of T-Ag. The gRNAs m1, m2, and m3 (respectively complimentary to SEQ ID NOS: 1, 5, and 9) were expressed in various combinations. As seen in FIG. 8, the induction of Cas9 expression by doxycycline along with the gRNAs severely decreased the number of colonies that are formed by these cells (FIGS. 8A-8D), indicative of T-Ag suppression by the Cas9 and the gRNAs in the cells. The combinations of gRNAs m1+m2, m1+m3, m2+m3, and m1+m2+m3, all reduced colony numbers. FIG. 8E illustrates a typical colony formation assay result.

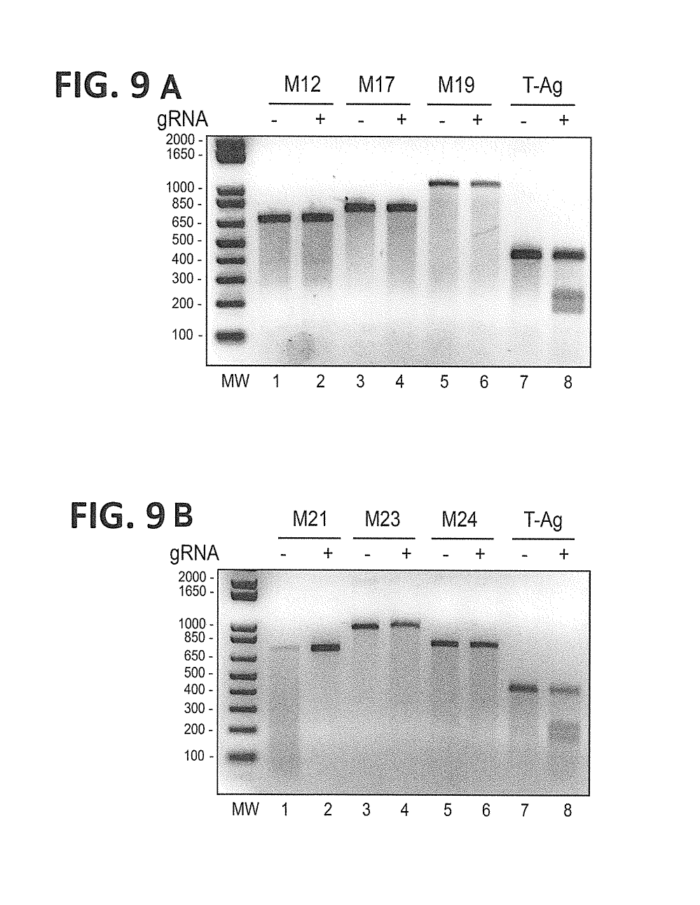

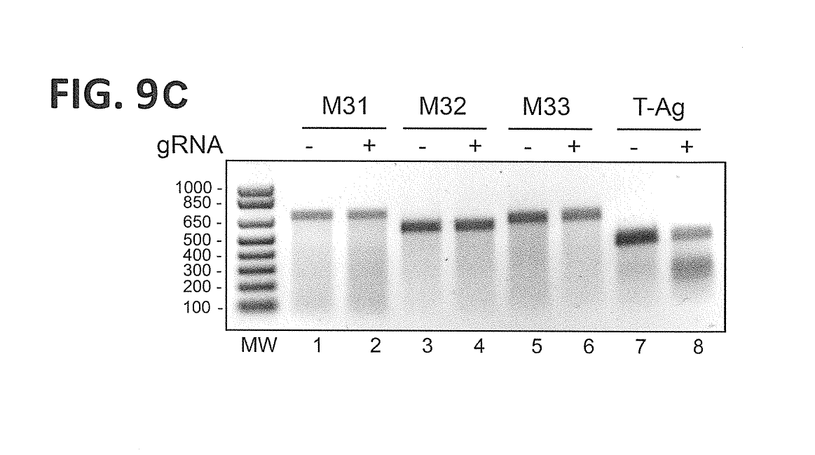

To assess any possible off-target events occurring in the cellular genome, stable Cas9 and gRNA expressing SVG-A cells were analyzed by SURVEYOR assay for InDel mutations in off-target genes. Human cellular genes with the highest degree of homology to each of the three motifs were identified by BLAST search at the NCB(website (www.ncbi.nlm.nih.gov/). For each motif, PCR products were amplified from the top three genes with the highest degree of homology and examined for InDel mutations using the SURVEYOR assay as described in Example 1. Results of analysis of motif 1 gRNA are shown in FIG. 9A. Results of analysis of motif 2 are shown in FIG. 9B. Results of analysis of motif 3 are shown in FIG. 9C. Amplification of T-Ag was used as a positive control in each experiment. Additional bands resulting from cleavage by SURVEYOR nuclease are shown by asterisks. In all cases, no cleavage of the off-target genes was detected, indicating the specificity of the CRISPR/Cas9.

The results disclosed in this example show that an inducible CRISPR system according to the present inventioncan cause drug-inducible ablation of the JCV T-Ag and alleviate the effects of JCV infection, without causing harmful off-target effects to similar normal genes. Taken together, the results of all of the preceding examples indicate that a CRISPR system according to the present invention is useful in eliminating actively replicating KV in PML patients, removing latent JCV from asymptomatic hosts of JCV, and preventing uninfected individuals at risk of JCV from acquiring the virus.

The invention has been described in an illustrative manner, and it is to be understood that the terminology that has been used is intended to be in the nature of words of description rather than of limitation. Obviously, many modifications and variations of the present invention are possible in light of the above teachings. It is, therefore, to be understood that within the scope of the appended claims, the invention can be practiced otherwise than as specifically described.

1. A composition for use in eliminating John Cunningham Virus (JCV) from a host cell infected with JCV, the composition comprising:
at least one isolated nucleic acid sequence encoding a Clustered Regularly Interspaced Short Palindromic Repeat (CRISPR)-associated endonuclease, and at least one guide RNA (gRNA) having a spacer sequence complementary to a target sequence in a JCV DNA.

2. The composition according to claim 1, wherein said at least one gRNA having a spacer sequence complementary to a target sequence in a JCV DNA is further defined as at least one gRNA having a spacer sequence complementary to a target sequence in the large T-antigen (T-Ag) encoding region of the JCV DNA.

3. The composition according to claim 2, wherein said at least one gRNA having a spacer sequence complementary to a target sequence in the T-Ag encoding region of the JCV DNA includes a gRNA having a spacer sequence complementary to a target sequence in the TM1 region of the T-Ag encoding region, a gRNA having a spacer sequence complementary to a target sequence in the TM2 region of the T-Ag encoding region, a gRNA having a spacer sequence complementary to a target sequence in the TM3 region of the T-Ag encoding region, or any combination of said gRNAs.

4. The composition according to claim 3 wherein said CRISPR-associated endonuclease is selected from a wild-type Cas9, a human-optimized Cas9, or a nickase mutant Cas9.

5. The composition according to claim 4, wherein said gRNA having a spacer sequence complementary to a target sequence in the TM1 region is gRNA m1, said gRNA having a spacer sequence complementary to a target sequence in the TM2 region is gRNA m2, and said gRNA having a spacer sequence complementary to a target sequence in the TM3 region is gRNA m3.

6. The composition according to claim 5, wherein said spacer sequence of said gRNA m1 is complementary to a target sequence including SEQ ID NOS: 1, 2, 3, or 4; said spacer sequence of said gRNA m2 is complementary to a target sequence including SEQ ID NOS: 5, 6, 7, or 8; and said spacer sequence of said gRNA m3 is complementary to a target sequence including SEQ ID NOS: 9, 10, 11, or 12.

7. The composition according to claim 1, wherein said CRISPR-associated endonuclease is Cpf1.

8. A method of eliminating John Cunningham Virus (JCV) from a host cell infected with JCV, including the steps of:
treating the host cell with a composition comprising a Clustered Regularly Interspaced Short Palindromic Repeat (CRISPR)-associated endonuclease, and at least one guide RNA (gRNA) having a spacer sequence that is complementary to a target sequence in a JCV DNA; and eliminating the JCV from the host cell.

9. The method according to claim 8, wherein the at least one gRNA having a spacer sequence complementary to a target sequence in a JCV DNA is further defined as at least one gRNA having a spacer sequence complementary to a target sequence in the large T-antigen (T-Ag) encoding region of the JCV DNA, and the method additionally includes, after the treating step, the step of deleting at least a segment of the JCV DNA situated in a coding region of T-Ag.

10. The method according to claim 9, wherein the at least one gRNA having a spacer sequence complementary to a target sequence in the T-Ag encoding region of the JCV DNA includes a gRNA having a spacer sequence complementary to a target sequence in the TM1 region of the T-Ag encoding region, a gRNA having a spacer sequence complementary to a target sequence in the TM2 region of the T-Ag encoding region, a gRNA having a spacer sequence complementary to a target sequence in the TM3 region of the T-Ag encoding region, or any combination of said gRNAs.

11. The method according to claim 10, wherein the CRISPR-associated endonuclease is selected from a wild-type Cas9, a human-optimized Cas9, or a nickase mutant Cas9.

12. The method according to claim 11, wherein the gRNA having a spacer sequence complementary to a target sequence in the TM1 region is gRNA m1, the gRNA having a spacer sequence complementary to a target sequence in the TM2 region is gRNA m2, and the gRNA having a spacer sequence complementary to a target sequence in the TM3 region is gRNA m3.

13. The method according to claim 12, wherein the spacer sequence of gRNA ml is complementary to a target sequence including SEQ ID NOS: 1, 2, 3, or 4; said spacer sequence of said gRNA m2 is complementary to a target sequence including SEQ ID NOS: 5, 6, 7, or 8; and said spacer sequence of said gRNA m3 is complementary to a target sequence including SEQ ID NOS: 9, 10, 11, or 12.

14. The method according to claim 8, wherein the CRISPR-associated endonuclease is Cpf1.

15. A vector composition for use in eliminating John Cunningham Virus (JCV) from a host cell infected with JCV, including:
at least one isolated nucleic acid sequence encoding a Clustered Regularly Interspaced Short Palindromic Repeat (CRISPR)-associated endonuclease, and
at least one guide RNA (gRNA) having a spacer sequence complementary to a target sequence in a JCV DNA,
said isolated nucleic acid sequences being included in at least one expression vector;
wherein said at least one expression vector induces the expression of said CRISPR-associated endonuclease and said at least one gRNA in a host cell.

16. The vector composition according to claim 15, wherein said at least one gRNA having a spacer sequence complementary to a target sequence in a JCV DNA is further defined as at least one gRNA having a spacer sequence complementary to a target sequence in the large T-antigen (T-Ag) encoding region of the JCV DNA.

17. The vector composition according to claim 16, wherein said at least one gRNA having a spacer sequence complementary to a target sequence in the T-Ag encoding region of the JCV DNA includes a gRNA having a spacer sequence complementary to a target sequence in the TM1 region of the T-Ag encoding region, a gRNA having a spacer sequence complementary to a target sequence in the TM2 region of the T-Ag encoding region, a gRNA having a spacer sequence complementary to a target sequence in the TM3 region of the T-Ag encoding region, or any combination of said gRNAs.

18. The vector composition according to claim 17, wherein said CRISPR-associated endonuclease is selected from a wild-type Cas9, a human-optimized Cas9, or a nickase mutant Cas9.

19. The vector composition according to claim 18, wherein said gRNA having a spacer sequence complementary to a target sequence in the TM1 region is gRNA m1, said gRNA having a spacer sequence complementary to a target sequence in the TM2 region is gRNA m2, and said gRNA having a spacer sequence complementary to a target sequence in the TM3 region is gRNA m3.

20. The composition according to claim 19, wherein said spacer sequence of said gRNA m1 is complementary to a target sequence including SEQ ID NOS: 1, 2, 3, or 4; said spacer sequence of said gRNA m2 is complementary to a target sequence including SEQ ID NOS: 5, 6, 7, or 8; and said spacer sequence of said gRNA m3 is complementary to a target sequence including SEQ ID NOS: 9, 10, 11, or 12.

21. The composition according to claim 15, wherein said CRISPR-associated endonuclease Cas9 is Cpf1.

22. The composition according to claim 15, wherein said expression vector is selected from the group consisting of a lentiviral expression vector, a drug inducible lentiviral expression vector, an adenovirus vector, an adeno-associated virus vector, a retroviral vector, a pox virus vector, and a plasmid vector.

23. The expression vector composition according to claim 15, wherein said CRISPR associated endonuclease and said at least one gRNA are incorporated into in a single expression vector.

24. The expression vector composition according to claim 15, wherein said CRISPR associated endonuclease and said at least one gRNA are incorporated into separate lentiviral expression vectors.

25. A method of preventing John Cunningham Virus (JCV) infection of cells of a patient at risk of JCV infection, including the steps of:
determining that a patient is at risk of JCV infection;
exposing cells of the patient at risk of JCV infection to an effective amount of an expression vector composition including an isolated nucleic acid encoding a Clustered Regularly Interspaced Short Palindromic Repeat (CRISPR)-associated endonuclease, and at least one isolated nucleic acid encoding at least one guide RNA (gRNA) including a spacer sequence complementary to a target sequence in a JCV DNA;
stably expressing the CRISPR-associated endonuclease and the at least one gRNA in the cells of the patient; and preventing JCV infection of the cells of the patient.

26. The method according to claim 25, wherein the at least one gRNA including a spacer sequence complementary to a target sequence in a JCV DNA is further defined as at least one gRNA including a spacer sequence complementary to a target sequence in the large T-antigen (TAg) encoding region of the JCV DNA.

27. A pharmaceutical composition including:
at least one isolated nucleic acid sequence encoding a Clustered Regularly Interspaced Short Palindromic Repeat (CRISPR)-associated endonuclease; and at least one isolated nucleic acid sequence encoding at least one guide RNA (gRNA) having a spacer sequence that is complementary to a target sequence in a John Cunningham Virus (JCV) genome;
said isolated nucleic acid sequences being included in at least one expression vector.

28. The pharmaceutical composition according to claim 27, wherein said at least one gRNA having a spacer sequence complementary to a target sequence in a JCV DNA is a further defined as at least one gRNA having a spacer sequence complementary to a target sequence in the large T-antigen (T-Ag) encoding region of the JCV DNA.

29. The pharmaceutical composition according to claim 28, wherein said at least one gRNA having a spacer sequence complementary to a target sequence in the T-Ag encoding region of the JCV DNA includes a gRNA having a spacer sequence complementary to a target sequence in the TM1 region of the T-Ag encoding region, a gRNA having a spacer sequence complementary to a target sequence in the TM2 region of the T-Ag encoding region, a gRNA having a spacer sequence complementary to a target sequence in the TM3 region of the T-Ag encoding region, or any combination of said gRNAs.

30. The pharmaceutical composition according to claim 29, wherein said CRISPR-associated endonuclease is selected from a wild-type Cas9, a human-optimized Cas9, or a nickase mutant Cas9.

31. The pharmaceutical composition according to claim 30, wherein said gRNA having a spacer sequence complementary to a target sequence in the TM1 region is gRNA m1, said gRNA having a spacer sequence complementary to a target sequence in the TM2 region is gRNA m2, and said gRNA having a spacer sequence complementary to a target sequence in the TM3 region is gRNA m3.

32. The pharmaceutical composition according to claim 31, wherein said spacer sequence of said gRNA M1 is complementary to a target sequence including SEQ ID NOS: 1, 2, 3, or 4; said spacer sequence of said gRNA m2 is complementary to a target sequence including SEQ ID NOS: 5, 6, 7, or 8; and said spacer sequence of said gRNA m3 is complementary to a target sequence including SEQ ID NOS: 9, 10, 11, or 12.

33. The pharmaceutical composition according to claim 27 wherein said CRISPR-associated endonuclease Cas9 is Cpf1.

34. The pharmaceutical composition according to claim 27, wherein said expression vector is selected from the group consisting of a lentiviral expression vector, a drug inducible lentiviral expression vector, an adenovirus vector, an adeno-associated virus vector, a retroviral vector, a pox virus vector, and a plasmid vector.

35. A method of treating a subject having a John Cunningham Virus (JCV) related disorder, including the step of administering to the subject an effective amount of a pharmaceutical composition according to claim 27.

36. The method according to claim 36, wherein the JCV-related disorder is progressive multifocal leukoencephalopathy (PML).

37. A kit for the treatment or prophylaxis of John Cunningham Virus (JCV) infection, including:
a measured amount of a composition comprising at least one isolated nucleic acid sequence encoding a Clustered Regularly Interspaced Short Palindromic Repeat (CRISPR)-associated endonuclease, and at least one nucleic acid sequence encoding one or more guide RNAs (gRNAs), wherein each of said one or more gRNAs includes a spacer sequence complementary to a target sequence in a JCV DNA; and
one or more items selected from the group consisting of packaging material, a package insert comprising instructions for use, a sterile fluid, a syringe and a sterile container.

38. The kit according to claim 37, wherein said expression vector is a lentiviral expression vector.

39. A method of eliminating a polyomavirus from a host cell infected with a polyomavirus, including the steps of:
treating the host cell with a composition comprising a Clustered Regularly Interspaced Short Palindromic Repeat (CRISPR)-associated endonuclease, and at least one guide RNA (gRNA) having a spacer sequence that is complementary to a target sequence in a polyomavirus DNA and eliminating the polyomavirus from the host cell.

40. The method according to claim 39, wherein the at least one gRNA having a spacer sequence complementary to a target sequence in a polyomavirus DNA is further defined as at least one gRNA having a spacer sequence complementary to a target sequence in the large T-antigen (T-Ag) encoding region of the polyomavirus DNA.


SEQUENCE LISTING (49 sequences)

SEQ ID NO: 1 - DNA, 31 bp, JC Virus
aaatgcaaagaactccaccctgatgaaggtg

SEQ ID NO: 2 - DNA, 34 bp, JC Virus
aaatgcaaagaactccaccctgatgaaggtgggg

SEQ ID NO: 3 - DNA, 31 bp, JC Virus
cacctttatcagggtggagttctttgcattt

SEQ ID NO: 4 - DNA, 34 bp, JC Virus
ccccacctttatcagggtggagttctttgcattt

SEQ ID NO: 5 - DNA, 40 bp, JC Virus
gatgaatgggaatcctggtggaatacatttaatgagaagt

SEQ ID NO: 6 - DNA, 43 bp, JC Virus
gatgaatgggaatcctggtggaatacatttaatgagaagtggg

SEQ ID NO: 7 - DNA, 40 bp, JC Virus
acttctcattaaatgtattccaccaggattcccattcatc

SEQ ID NO: 8 - DNA, 43 bp, JC Virus
cccacttctcattaaatgtattccaccaggattcccattcatc

SEQ ID NO: 9 - DNA, 33 bp, JC Virus
aaggtactggctattcaaggggccaatagacag

SEQ ID NO: 10 - DNA, 36 bp, JC Virus
aaggtactggctattcaaggggccaatagacagtgg

SEQ ID NO: 11 - DNA, 33 bp, JC Virus
ctgtctattggccccttgaatagccagtacctt

SEQ ID NO: 12 - DNA, 36 bp, JC Virus
ccactgtctattggccccttgaatagccagtacctt

SEQ ID NO: 13 - DNA, 50 bp, JC Virus
cttgtcttcgtccccacctttatcagggtggagttctttgcattttttca

SEQ ID NO: 14 - DNA, 50 bp, JC Virus
tgaaaaaatgcaaagaactccaccctgatgaaggtggggacgaagacaag

SEQ ID NO: 15 - DNA, 50 bp, JC Virus
ttcatcccacttctcattaaatgtattccaccaggattcccattcatctg

SEQ ID NO: 16 - DNA, 50 bp, JC Virus
cagatgaatgggaatcctggtggaatacatttaatgagaagtgggatgaa

SEQ ID NO: 17 - DNA, 50 bp, JC Virus
tttgccactgtctattggccccttgaatagccagtaccttttttttggaa

SEQ ID NO: 18 - DNA, 50 bp, JC Virus
ttccaaaaaaaaggtactggctattcaaggggccaatagacagtggcaaa

SEQ ID NO: 19 - DNA, 37 bp, Artificial Sequence
  TM1 fwd primer px260
aaacaaatgcaaagaactccaccctgataaaggtggt

SEQ ID NO: 20 - DNA, 37 bp, Artificial Sequence
  TM1 rev primer px260
taaaaccacctttatcagggtggagttctttgcattt

SEQ ID NO: 21 - DNA, 46 bp, Artificial Sequence
  TM2 fwd primer px260
aaacgatgaatgggaatcctggtggaatacatttaatgagaagtgt

SEQ ID NO: 22 - DNA, 46 bp, Artificial Sequence
  TM2 rev primer px260
taaaactcttctcattaaatgtattccaccaggattcccattcatc

SEQ ID NO: 23 - DNA, 40 bp, Artificial Sequence
  TM2 fwd primer px260
aaacaaaggtactggctattcaaggggccaatagacaggt

SEQ ID NO: 24 - DNA, 40 bp, Artificial Sequence
  TM3 rev primer px260
taaaacctgtcatttggccccttgaatagccagtaccttt

SEQ ID NO: 25 - DNA, 24 bp, Artificial Sequence
  TM1 fwd primer px330
caccgcaccaccctgataaaggtg

SEQ ID NO: 26 - DNA, 24 bp, Artificial Sequence
  TM1 rev primer px330
aaaccacctttatcagggtggtgc

SEQ ID NO: 27 - DNA, 24 bp, Artificial Sequence
  TM2 fwd primer px330
caccgaatacatttaatgagaagt

SEQ ID NO: 28 - DNA, 24 bp, Artificial Sequence
  TM2 rev primer px330
aaacacttctcattaaatgtattc

SEQ ID NO: 29 - DNA, 24 bp, Artificial Sequence
  TM3 fwd primer px330
caccgtcaaggggccaatagacag

SEQ ID NO: 30 - DNA, 24 bp, Artificial Sequence
  TM3 rev primer px330
aaacctgtctattggccccttgac

SEQ ID NO: 31 - DNA, 51 bp, JC Virus
cttgtcttcgtccccaccctttatcagggtggagttctttgcattttttca

SEQ ID NO: 32 - DNA, 51 bp, JC Virus; identical to SEQ ID NO: 31

SEQ ID NO: 33 - DNA, 51 bp, JC Virus
cttgtgttcgtccccaccctttatcagggtggagttctttgcattttttca

SEQ ID NO: 34 - DNA, 48 bp, JC Virus
cttgtcttcgtccccatttatcagggtggagttctttgcattttttca

SEQ ID NO: 35 - DNA, 49 bp, JC Virus
ttcatcccactctcattaaatgtattccaccaggattcccattcatctg

SEQ ID NO: 36 - DNA, 48 bp, JC Virus
ttcatcccatctcattaaatgtattccaccaggattcccattcatctg

SEQ ID NO: 37 - DNA, 49 bp, JC Virus; identical to SEQ ID NO: 35

SEQ ID NO: 38 - DNA, 49 bp, JC Virus; identical to SEQ ID NO: 35

SEQ ID NO: 39 - DNA, 48 bp, JC Virus
tttgccactgtattggccccttgaatagccagtaccttttttttggaa

SEQ ID NO: 40 - DNA, 49 bp, JC Virus
tttgccactgttattggccccttgaatagccagtaccttttttttggaa

SEQ ID NO: 41 - DNA, 51 bp, JC Virus
tttgccactggtctattggccccttgaatagccagtaccttttttttggaa

SEQ ID NO: 42 - DNA, 38 bp, Artificial Sequence
  primer
tatgggcccacgcgtgagggcctatttcccatgattcc

SEQ ID NO: 43 - DNA, 38 bp, Artificial Sequence
  primer
tgtggatcctcgaggcgggccatttaccgtaagttatg

SEQ ID NO: 44 - DNA, 22 bp, Artificial Sequence
  primer
gcttatgccatgccctgaaggt

SEQ ID NO: 45 - DNA, 23 bp, Artificial Sequence
  primer
atggacaaagtgctgaataggga

SEQ ID NO: 46 - DNA, 21 bp, Artificial Sequence
  primer
ctctggtcatgtggatgctgt

SEQ ID NO: 47 - DNA, 23 bp, Artificial Sequence
  primer
atggacaaagtgctgaataggga

SEQ ID NO: 48 - DNA, 22 bp, Artificial Sequence
  primer
gcttatgccatgccctgaaggt

SEQ ID NO: 49 - DNA, 21 bp, Artificial Sequence
  primer
acagcatccacatgaccagag